(19) United States
(12) Patent Application Publication
(10) Pub. No.: US 2019/0237203 A1
(43) Pub. Date: Aug. 1, 2019

(54) FACILITATING SELF-SCHEDULING OF MEDICAL APPOINTMENTS

(71) Applicant: University Hospitals Cleveland Medical Center, Cleveland, OH (US)

(72) Inventors: Craig Schwabl, University Heights, OH (US); Cynthia Zelis, Strongsville, OH (US); Andrew Laytin, Shaker Heights, OH (US)

(21) Appl. No.: 15/881,335

(22) Filed: Jan. 26, 2018

(51) Int. Cl. G16H 80/00; G06Q 10/10; G06F 21/62

(52) U.S. Cl. CPC G16H 80/00; G06K 7/1413; G06F 21/6245; G06Q 10/1095

(57) ABSTRACT

Systems, methods and computer readable media are provided that facilitate self-scheduling of medical appointments, particularly specialty referral appointments. In an embodiment, a method can include receiving, by a system comprising a processor, a referral order identifier for a medical referral ordered for a patient. The method can further include determining, by the system, referral order information associated with the referral order in an electronic medical record for the patient based on the referral order identifier, wherein the referral order information identifies a type of medical appointment recommended for the patient. The method can further include, facilitating, by the system, scheduling the type of medical appointment for the patient with a remote medical scheduling system based on the referral order information, and generating, by the system, a graphical user interface that facilities the scheduling of the type of medical appointment.

### TECHNICAL FIELD

This application generally relates to computer processing systems, computer-implemented methods, apparatus and/or computer program products that facilitate self-scheduling of medical appointments, particularly specialty referral appointments.

### BACKGROUND

In the United States, more than a third of patients are referred to a specialist each year, and specialist visits constitute more than half of outpatient visits. Despite the frequency of referrals and the importance of the specialty-referral process, the process itself has been a long-standing source of frustration among both primary care physicians (PCPs) and specialists. For example, 50% of referrals never result in a specialist visit, and 25% of referrals send patients to the wrong specialist. Further, an alarming 20% of malpractice claims are attributed to missed or delayed diagnosis that could have been avoided if the patient was referred to and seen by the correct specialist. These frustrations, along with a desire to lower costs, have led to the need for strategies to improve the specialty-referral process.

### BRIEF DESCRIPTION OF THE DRAWINGS

Numerous aspects, embodiments, objects and advantages of the present invention will be apparent upon consideration of the following detailed description, taken in conjunction with the accompanying drawings, in which like reference characters refer to like parts throughout, and in which:

FIG. 1 illustrates an example system that facilitates self-scheduling of medical appointments in accordance with various aspects and embodiments described herein;

FIG. 2 presents an example client appointment management component that facilitates self-scheduling of medical appointments in accordance with various aspects and embodiments described herein;

FIGS. 3A-3L presents various graphical user interfaces (GUI)s associated with an application that facilitates self-scheduling of medical appointments in accordance with various aspects and embodiments described herein;

FIG. 4 presents another example client appointment management component that facilitates self-scheduling of medical appointments in accordance with various additional embodiments described herein;

FIG. 5 presents another example client appointment management component that facilitates self-scheduling of medical appointments in accordance with various additional embodiments described herein;

FIG. 6 presents another example client appointment management component that facilitates self-scheduling of medical appointments in accordance with various additional embodiments described herein;

FIG. 7 provides a high level flow diagram of an example computer-implemented method that facilitates self-scheduling of medical appointments in accordance with various aspects and embodiments described herein;

FIG. 8 provides a flow diagram of another example computer-implemented method that facilitates self-scheduling of medical appointments in accordance with various aspects and embodiments described herein;

FIG. 9 provides a flow diagram of another example computer-implemented method that facilitates self-scheduling of medical appointments in accordance with various aspects and embodiments described herein;

FIG. 10 is a schematic block diagram illustrating a suitable operating environment in accordance with various aspects and embodiments.

FIG. 11 is a schematic block diagram of a sample-computing environment in accordance with various aspects and embodiments.

### DETAILED DESCRIPTION

The following detailed description is merely illustrative and is not intended to limit embodiments and/or application or uses of embodiments. Furthermore, there is no intention to be bound by any expressed or implied information presented in the preceding Background or Summary sections, or in the Detailed Description section.

The subject disclosure is directed to computer processing systems, computer-implemented methods, apparatus and/or computer program products that facilitate self-scheduling of medical appointments, particularly specialty referral appointments. In particular, the proposed self-scheduling techniques provide a frictionless integrated web and mobile application allowing patients to schedule their provider ordered new patient specialty referrals. The disclosed techniques provide several advantages of the existing complicated specialty referral process. For example, with the disclosed techniques, a patient can efficiently and easily schedule a specialty referral appointment at their own convenience with a service provider that has been specifically selected to suit the patient's needs. As result, the disclosed techniques maximize referral order capture, enhance clinical integration, improve patient experience and safety, and improve overall operational efficiency associated with scheduling specialty appointments.

In one or more embodiments, a system is disclosed that comprises a memory that stores computer executable components and a processor that executes computer executable components stored in the memory. The computer executable components can comprise a reception component configured to receive a referral order identifier for a medical referral ordered for a patient, and a order identification component configured to access an electronic medical record for the patient and retrieve referral order information associated with the referral order based on the referral order identifier, wherein the referral order information identifies a type of medical appointment recommended for the patient. The components can further comprise a scheduling component configured to access a remote medical scheduling system to facilitate scheduling the type of medical appointment for the patient based on the referral order information, and an interface component configured to generate a graphical user interface that facilities the scheduling of the type of medical appointment.

In some implementations, the scheduling component can comprise a provider identification component configured to determine provider information identifying one or more providers that are qualified to perform the type of medical appointment based on the referral order information and qualification information for the one or more providers. The scheduling component can further be configured to schedule the type of medical appointment for the patient with the remote medical scheduling system based on reception of input selecting a provider of the one or more providers. In one or more implementations, the scheduling component further comprises a filter component configured to select a subset of the one or more providers for recommending to the patient based on one or more criterion. For example, the one or more criterion can comprise availability of the one or more providers within a defined time frame. In another example, the one or more criterion can comprise respective locations of the one or more providers relative a location associated with the patient. In another example, the one or more criterion can comprise respective types of insurance coverage accepted by the one or more providers relative a type of insurance coverage used by the patient. In yet another example, the one or more criterion comprise a preferred characteristic for the subset of medical providers, wherein the preferred characteristic is defined in preference information for the patient.

In some implementations, the scheduling component further comprises an availability component configured to determine availability information regarding available appointment slots at which the one or more providers can perform the type of medical appointment. With these implementations, the scheduling component can be configured to schedule the type of medical appointment for the patient with the remote medical scheduling system based on reception of input selecting an appointment slot of the available appointment slots for a selected provider of the one or more providers.

In another embodiment, a method can include receiving, by a system comprising a processor, a referral order identifier for a medical referral ordered for a patient. The method can further include determining, by the system, referral order information associated with the referral order in an electronic medical record for the patient based on the referral order identifier, wherein the referral order information identifies a type of medical appointment recommended for the patient. The method can further include, facilitating, by the system, scheduling the type of medical appointment for the patient with a remote medical scheduling system based on the referral order information, and generating, by the system, a graphical user interface that facilities the scheduling of the type of medical appointment.

In another embodiment, a machine-readable storage medium, comprising executable instructions that, when executed by a processor, can facilitate performance of operations. These operations can comprise receiving a referral order identifier for a medical referral ordered for a patient, and determining referral order information associated with the referral order in an electronic medical record for the patient based on the referral order identifier, wherein the referral order information indicates a clinical condition of the patient. The operations can further comprise, determining provider information identifying one or more providers that are qualified to treat the medical condition, receiving a request to schedule a medical appointment for the patient with a selected provider of the one or more providers, and based on the receiving request, scheduling the medical appointment for the patient using at remote medical scheduling system.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

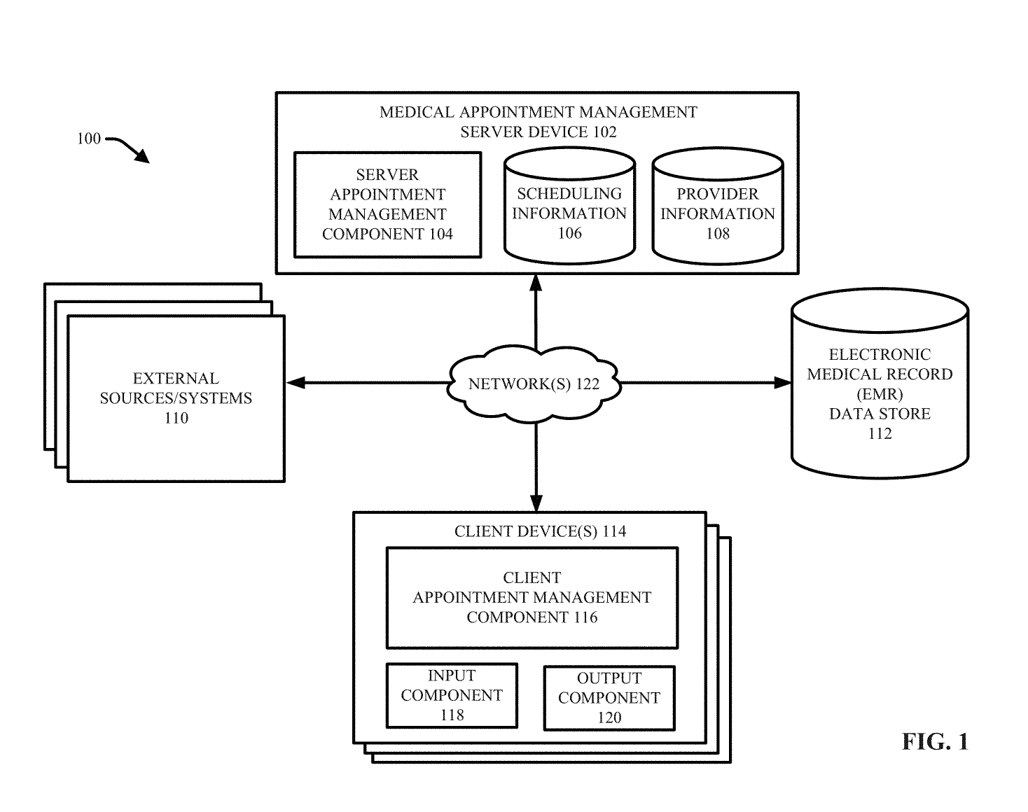

Referring now to the drawings, with reference initially to FIG. 1, presented is a high level block diagram of an example system 100 that facilitates self-scheduling of medical appointments in accordance with aspects and embodiments described herein. Aspects of systems, apparatuses or processes explained in this disclosure can constitute machine-executable components embodied within machine(s), (e.g. embodied in one or more computer readable mediums (or media) associated with one or more machines). Such components, when executed by the one or more machines, e.g. computer(s), computing device(s), virtual machine(s), etc. can cause the machine(s) to perform the operations described.

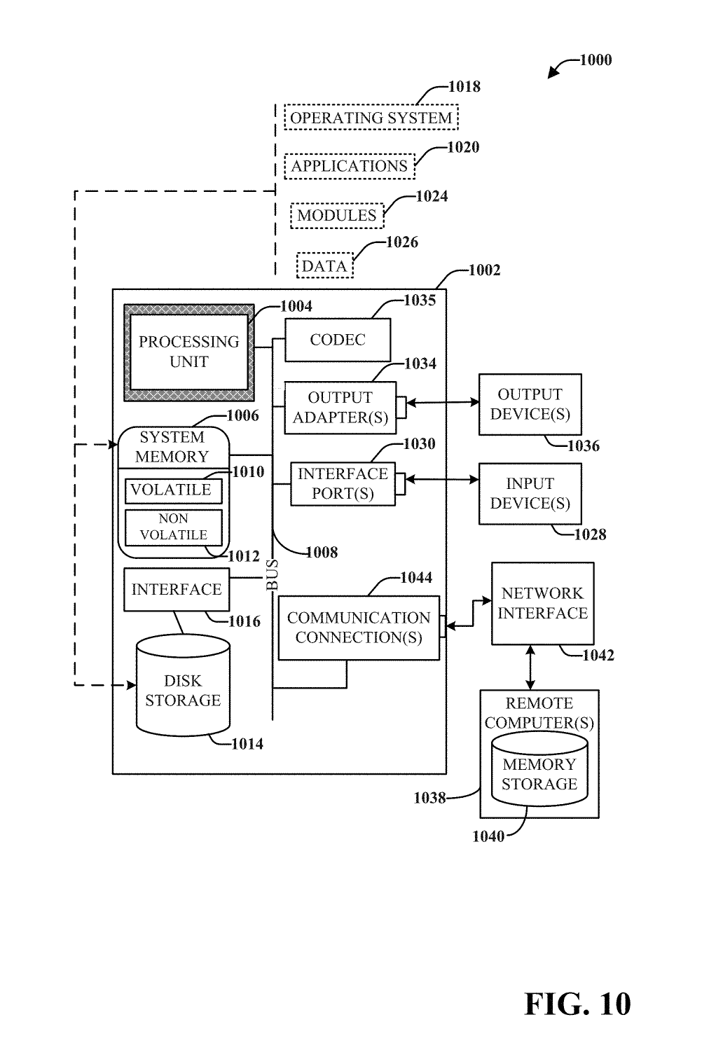

System 100 can include a medical appointment management server device 102, one or more external sources/systems 110, an EMR data store 112, and one or more client devices 114. The medical appointment management server device 102 can include one or more (communicatively coupled) computing devices (e.g. physical devices and/or virtual devices or machines). In this regard, the one or more computing devices of the medical appointment management server device 102 can include or be communicatively coupled to at least one memory that stores executable components (e.g. the server appointment management component 104) and at least one processor that executes the executable components stored in the memory. The medical appointment management server device 102 can also include or otherwise be communicatively coupled to one or more data stores including scheduling information 106 and provider information 108 that facilitates self-scheduling of medical appointments. In some implementations, the medical appointment management server device 102 can also include the EMR data store 112. Likewise, the one or more client devices 114 can respectively include at least one memory that stores executable components (e.g. the client appointment management component 116), and at least one processor that executes the executable components stored in the memory. Examples of said processors and memories, as well as other suitable computer or computing-based elements that can be included with the medical appointment management server device 102 and/or the one or more client devices 114, can be found infra with reference to FIG. 10.

The one or more client devices 114 can include any suitable computing device associated with a user and configured to facilitate accessing and employing the information and services provided by the medical appointment management server device 102. For example, the one or more client devices 114 can include a mobile phone, a smartphone, a tablet personal computer (PC), a personal digital assistant PDA, a desktop computer, a laptop computer, a television, an Internet enabled television, a heads-up display (HUD), virtual reality (VR) headset, augmented reality (AR) headset, or another type of wearable computing device. The respective client devices 14 can include suitable hardware and software that facilitates receiving and interpreting user input, displaying or rendering information to the user, and communicating with other devices (e.g. the medical appointment management server device 102, the one or more external sources/systems 110, the EMR data store 112 etc.). For example, the respective client devices 114 can include an input component 118 to facilitate receiving and interpreting user input. The input component 118 can include suitable input hardware (and associated software), including but not limited to: a touchscreen, hard or soft buttons, a keyboard, a keypad, a mouse, a joystick, voice/audio input device, a motion/gesture input device, a camera, a scanner and the like. The respective client devices 114 can also include an output component 120 that includes hardware, software or a combination of hardware and software that provides output information to a user. For example, the output component 120 can include a display screen, a speaker, a vibration apparatus, and the like. The respective client devices 114 can also include suitable communication hardware and associated software (e.g. a central processing unit (CPU), a transmitter, a receiver, a transceiver, a decoder/encoder, etc.) to facilitate wired and/or wireless communication between the client device 114 and other devices (e.g. the medical appointment management server device 102, the one or more external sources/systems 110, the EMR data store 112 and the like).

The various devices, systems/sources, and components of system 100 can be communicatively connected either directly or via one or more networks 122. Such network(s) can include wired and wireless communication networks, including but not limited to, a cellular network, a wide area network (WAN) (e.g. the Internet), a local area network (LAN), or a personal area network (PAN) or the like. For example, a client device 114 can communicate with the medical appointment management server device 102, one or more external sources/systems 110, and/or the EMR data store 112 (and vice versa) using virtually any desired wired or wireless communication technology, including but not limited to: a near filed communication (NFC) based protocol, a BLUETOOTH® technology-based protocol, a ZigBee® based protocol, a Wi-Fi protocol, an RF based communication protocol, an IP based communication protocol, a cellular communication protocol, a UWB technology-based protocol, or other forms of communication including both proprietary and non-proprietary communication protocols. In an aspect, one or more devices, systems/sources, and components of system 100 can be configured to interact via disparate networks/communication technologies.

In accordance with various embodiments, the medical appointment management server device 102 can be configured to provide or facilitate various services associated with self-scheduling of medical appointments by patients (or authorized agents of the patients'). For example, the medical appointment management server device 102 can include server appointment management component 104 to function as a centralized medical appointment scheduler and manger for a plurality of different medical service providers and patients. In some implementations, the respective service providers and/or patients can be associated with a single medical organization, system or hospital. In other implementations, the respective service providers and/or patients can be associated with different medical organizations and/or independent medical practices. In either of these implementations, the server appointment management component 104 can provide appointment scheduling tools and features as a hosted software solution accessible via a web browser and/or mobile application. This facilitates appointment scheduling over the web by providing patients access to medical service provider's schedules and allowing patients to make appointments online at their convince. In this regard, using a client device 114, a patient (or authorized agent of the patient), can access information provided by the medical appointment management server device 102 regarding service providers that fit the patient's needs and the available appointments for the respective service providers. For example, the patient can interface with the medical appointment management server device 102 online via a website or mobile application provided and/or serviced by the medical appointment management server device 102. The patient can further select and request to book a particular appointment with a specific service provider at a set time and location, and the server appointment management component 104 can receive and processes the requests to automatically book the appointment.

In some embodiments the server appointment management component 104 can be or include existing medical scheduling/management software employed by a medical organization and/or one or more of the medical service providers, such as ATHENAHEALTH™, SOARIAN™, and the like. According to these embodiments, the respective client devices 114 can be configured to interface with the server appointment management component 104 using a client appointment management component 116 and the suitable application program interfaces (APIs) for the existing medical scheduling/management software. In other embodiments, system 100 can employ a third party medical appointment scheduling/management system that is external to the medical appointment management server device 102. For example, the third party scheduling/management system can be provided at an external source or system of the one or more external sources/systems 110. With these embodiments, the server appointment management component 104 can be configured to interface with the third party medical appointment scheduling/management system using the appropriate API to facilitate scheduling medical appointments.

In the embodiment shown, the various medical appointment scheduling/management services facilitated by system 100 can be provided by the medical appointment management server device 102 using a network accessible platform (e.g., a website or mobile application). For example, in some implementations, these services can be embodied in a software application, such as a web-application, a cloud-application, a thin client application, a thick client application, a native client application, a hybrid client application, or the like. In this regard, the medical appointment management server device 102 and the one or more client devices 114 can be configured to operate in client/server relationship. According to these embodiments, the medical appointment management server device 102 can be configured to perform various back-end processing functions associated with self-scheduling of medical appointments using server appointment management component 104. Likewise, the one or more client devices 114 can respectively include a client appointment management component 116 configured to perform various front-end processing functions associated with self-scheduling of medical appointments. However, it should be appreciated however that system 100 is not limited to this architectural configuration. For example, in some embodiments, one or more features and functionalities of the server appointment management component 104 can be provided at the client appointment management component 116. Likewise, one or more features and functionalities of the client appointment management component 116 can be provided at the server appointment management component 104.

In one or more exemplary embodiments, system 100 can be particularly configured to facilitate self-scheduling of specialty referral appointments. In medicine, a referral is the recommendation of a medical practitioner to send a patient to another medical practitioner (e.g., a specialist) for consultation or a health care service that the referring practitioner believes is necessary but is not prepared or qualified to provide. In health maintenance organizations (HMOs) and other managed care schemes, a referral is usually required for a patient to see any practitioner or specialist other than the patient's PCP in order for the service to be financially covered. A specialty referral is the interface between the referring provider and a specialist. While the referring provider can be any type of provider, various exemplary embodiments of the disclosed techniques for self-scheduling medical appointments are exemplified with reference to the referring provider being the patient's PCP. However, it should be appreciated that a specialty referral can include a referral from one specialist to another, also known as a cross-referral.

In various healthcare systems, when a patient receives a referral from a medical practitioner, the medical practitioner generates a referral order authorizing the referral. For example, in some scenarios, the referring clinician can provide the patient with a written referral order. In other scenarios, the referring clinician can provide the patient with a printed order form. Still in other scenarios, the referring clinician can generate an electronic referral order and associate it with an electronic account that is accessible to the patient. Regardless of the manner in which the patient is provided with a referral order, in accordance with one or more embodiments of the subject disclosure, the referring clinician (or an authorized agent of the referring clinician) can further enter the referral order into an electronic filing system that records the referral order for the patient. For example, in the embodiment shown, the referral order can be entered into the patient's electronic medical record (EMR) or ambulatory medical record (aEMR) and stored in the EMR data store 112.

The EMR data store 112 can include EMRs and/or aEMR for a plurality of patients. In some implementations, the EMR data store 112 can include information for patients associated with particular medical system or organization. In other implementations, the EMR data store 112 can include information for patients of disparate medical systems or organizations. In this regard, the EMR data store can also include electronic health records (EHRs) for the patients. An aEMR is similar to an EMR but while EMRs keep track of inpatient care (surgeries and care that require spending overnight or longer in a hospital), aEMRs generally provided information regarding medical procedures and care that do not result in an overnight stay in a hospital or that are given in non-hospital settings such as urgent care clinics, physicians' offices and at-home medical care. In combination with EMRs, aEMRs and EHRs allow a clinician to view a patient's complete and accurate medical history.

In one or more embodiments, in association with entry of referral orders into the patient's EMR, aEMR, (or an alternative electronic record for the patient), the referral order can be associated with a unique identifier (e.g., a unique number) that identifies the referral order and the patient to which it is assigned. In some embodiments, the unique identifier for the referral order can be embodied in a barcode that is printed on a referral order form that is provided to the patient. According to these embodiments, the barcode can be scanned to identify the referral order and its associated information in the patient's EMR. The referral order can also include information regarding the reason for the referral and/or the type of medical appointment or service recommended for the patient. For example, in some implementations, the referral order can include information identifying the clinical reason for the referral and/or information identifying the clinical condition or conditions of the patient for which the order is based. In some implementations, the referring provider can also recommend a particular provider to the patient. With these implementations, the referral order can also include information identifying the recommended provider or providers.

In various embodiments, systems 100 facilitates self-scheduling of specialty referral appointments in an efficient manner based on providing the client appointment management component 116 and/or the server appointment management component 104 access to a patient's referral order information in the EMR data store 112. In this regard, in one or more embodiments, the client appointment management component 116 can be configured to receive input at the client device 114 comprising a referral order identifier for a medical referral ordered for a patient. Based on the received order identifier, the client appointment management component 116 and/or the server appointment management component 104 can be configured to access the patient's EMR and retrieve the referral order information associated with the referral order corresponding to the referral order identifier. The client appointment management component 116 can further be configured to interface with the server appointment management component 104 to facilitate scheduling the specialty referral appointment for the patient based on the referral order information. For example, based on the referral order information identifying a type of specialist recommended for the patient or a medical condition of the patient, the client appointment management component 116 and/or the server appointment management component 104 can determine and provide the patient with information identifying suitable service providers that can satisfy the referral order. The client appointment management component 116 and/or the server appointment management component 104 can also determine and provide the patient with information regarding available appointments of the respective service providers, locations of the respective service providers, reviews of the respective service providers, etc., and allow the patient to choose a particular service provider, location and time slot for the appointment. Based on user input at the client device 114 selecting the appointment for booking, the client appointment management component 116 can further interface with the server appointment management component 104 to complete booking and schedule the appointment.

In order to facilitate scheduling medical appointment for patients, the medical appointment management server device 102 can include, or otherwise provide the server appointment management component 104 and/or the client appointment management component 116 access to, scheduling information 106 for the respective medical service providers and provider information 108 for the respective providers. The scheduling information 106 can include various types of up to date information for the respective service providers that is relevant to scheduling patient specialty appointments therewith. For example, the scheduling information 106 can identify available appointments for the respective service providers including information regarding date, time, location, duration, and the like. In some implementation the scheduling information 106 can also include information regarding special requirements or restrictions for the appointments. The scheduling information 106 can also included information regarding scheduled appointments.

The provider information 108 can include descriptive information for the respective service providers regarding the clinical services that they are capable and qualified to perform. For example, the provider information can identify, for each service provider, their specialty or practice area, the types of services or procedures they can perform, the types of conditions they can treat, and the like. In some embodiments, the provider information 108 can also describe various professional attributes of the respective clinicians, such as those that may be found on a professional resume (e.g., education, employment history, performance history, accolades, etc.). The provider information can also include demographic information for the respective providers, reviews or ratings provided for the respective service providers, any other relevant information about the respective service providers that may be useful in helping a patient determine whether to schedule an appointment with a particular service provider.

In the embodiment shown, the scheduling information 106 and the provider information 108 are located at the medical appointment management server device 102. However, it should be appreciated that the location of the scheduling information 106 and the provider information 108 can be external to the medical appointment management server device 102 and communicatively coupled to the server appointment management component 104. For example, in some implementations, the scheduling information for a particular service provider can be stored locally at an internal system associated with the service provider (e.g., an external system/source associated with the service provider). According to these implementations, the server appointment management component 104 can access and/or retrieve the scheduling information for that service provider at the service provider's internal system/source. In some embodiments, the server appointment management component 104 can be configured to regularly interface with external scheduling systems for service providers to retrieve up to date scheduling information for the service providers and update the scheduling information 106 accordingly.

In addition to the patient EMR and/or aEMR provided in the EMR data store 112, the scheduling information, 106 and the provider information 108, in some embodiments, system 100 can include additional information about patients that can be used by the server appointment management component 104 and/or the client appointment management component 116 to facilitate personalized services associated with scheduling and managing medical appointments. For example, in some embodiments, the respective patients can be associated with user accounts or profiles that provide information about the patients regarding their clinical preferences, such as information regarding characteristics of service providers they prefer (e.g., gender, age, qualifications, time in practice, etc.) and characteristics of appointments they prefer (e.g., location, drive time, time of day, day of week, etc). In another example, a patient's profile information can identify specific service providers (e.g., by name) that they prefer as well as those which they don't prefer. In some embodiments, a patient's profile information can also include demographic information for the patient. Further, in various embodiments, a patient's profile can include insurance information for the patient (e.g., insurance provider and coverage plan). In some implementations, the patient profile information can be associated with the respective patients' EMRs provided in the EMR data store 112. In other embodiments, the patient profile information can be stored locally at the medical appointment management server device 102 or the client device 114. Still in other embodiments, the patient profile information can be located at an external source or system (e.g., of the one or more external sources/systems 110) that is accessible to the server appointment management component 104 and/or the client appointment management component.

In various embodiments in which a patient is associated with a profile and/or account including information regarding patient preferences, demographic information and the like, the patient can access and edit the patient profile information at will via a suitable log-in, authorization process. Further, in implementation in which the patient is provided recommended service providers and/or appointment slots based on the patient's existing profile information, the patient can further control tailoring of the results by providing new selection/filtering criteria that differs from what is included in the patient's profile information. The patient can also provide input that identifies which criteria to emphasize or weigh more heavily than others when receiving recommended providers and/or appointment slots. For example, the patient may indicate that availability within the next three days is more important than whether the patient sees a physician located near her home location.

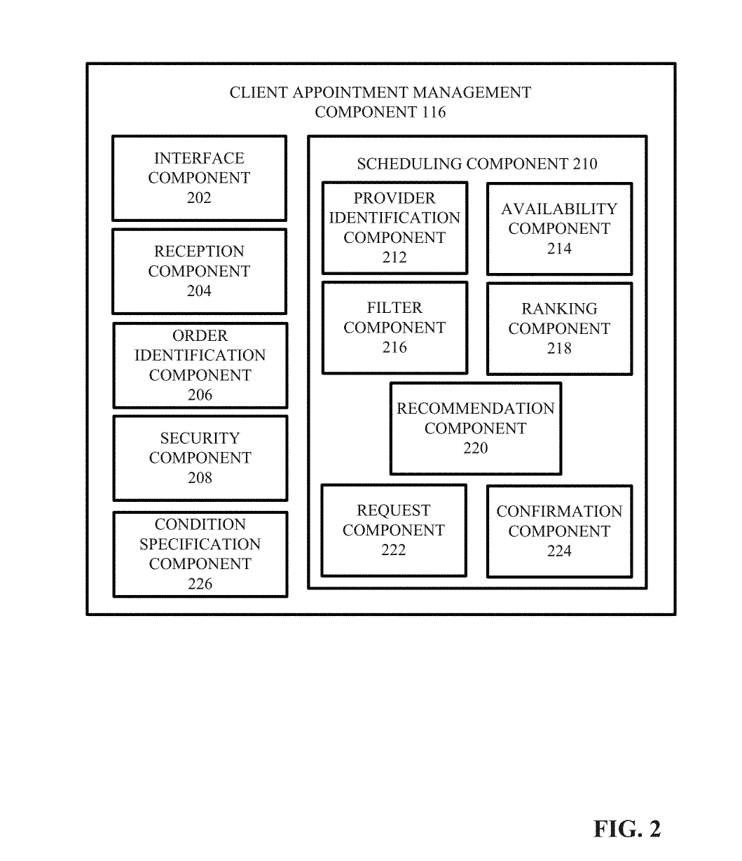

Various additional features and functionalities of the client appointment management component 116 and the server appointment management component 104 are described infra with reference to FIGS. 2-6.

FIG. 2 presents an example client appointment management component 116 in accordance with various aspects and embodiments described herein. In the embodiment shown, the client appointment management component can include interface component 202, reception component 204, order identification component 206, security component 208, condition specification component 226, and scheduling component 210.

With reference to FIGS. 1 and 2, in various embodiments, the client appointment management component 116 corresponds to a client application (e.g., a thin client application, web-application, a mobile application, etc.), that is provided at the client device 114 and configured to interface with the server appointment management component 104 to facilitate operations of the associated components (e.g., the interface component 202, the reception component 204, the order identification component 206, the security component 208, and the scheduling component 210). However, it should be appreciated that one or more of the operations provided by the various components of the client appointment management component 116 can be distributed between the client appointment management component 116 and the server appointment management component 104. For example, in some embodiments, one or more of the interface component 202, the reception component 204, the order identification component 206, the security component 208, and the scheduling component 210 can be provided with the server appointment management component 104 and executed by the medical appointment management server device 102. Repetitive description of like elements employed in respective embodiments is omitted for sake of brevity.

The interface component 202 can be configured to generate one or more GUIs for presentation at the client device 114 (e.g., via a display) that facilitate self-scheduling and managing of medical appointments, particularly specialty referral appointments, using the client appointment management component 116. For example, the one or more GUIs can allow a patient (or authorized agent of the patient) to access and view referral orders issued for the patient, select a referral order for scheduling, search for and view suitable service providers and available appointments for the referral order, filter appointments and service providers based on one or more filter criteria, select a specific service provider and appointment for scheduling, and the like.

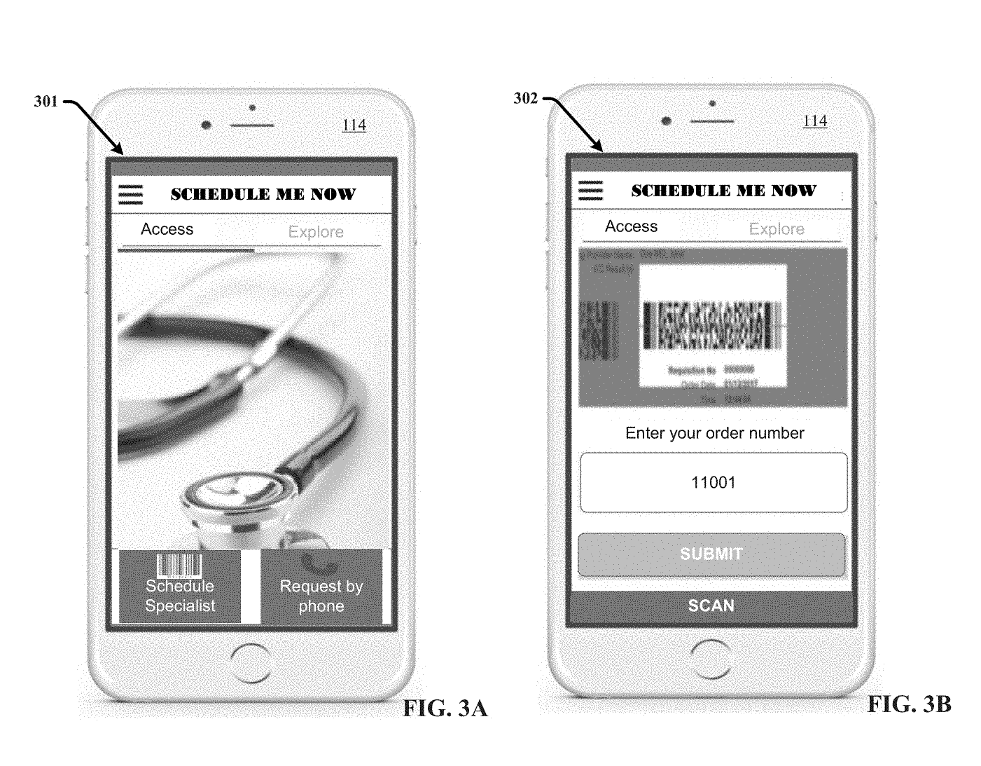

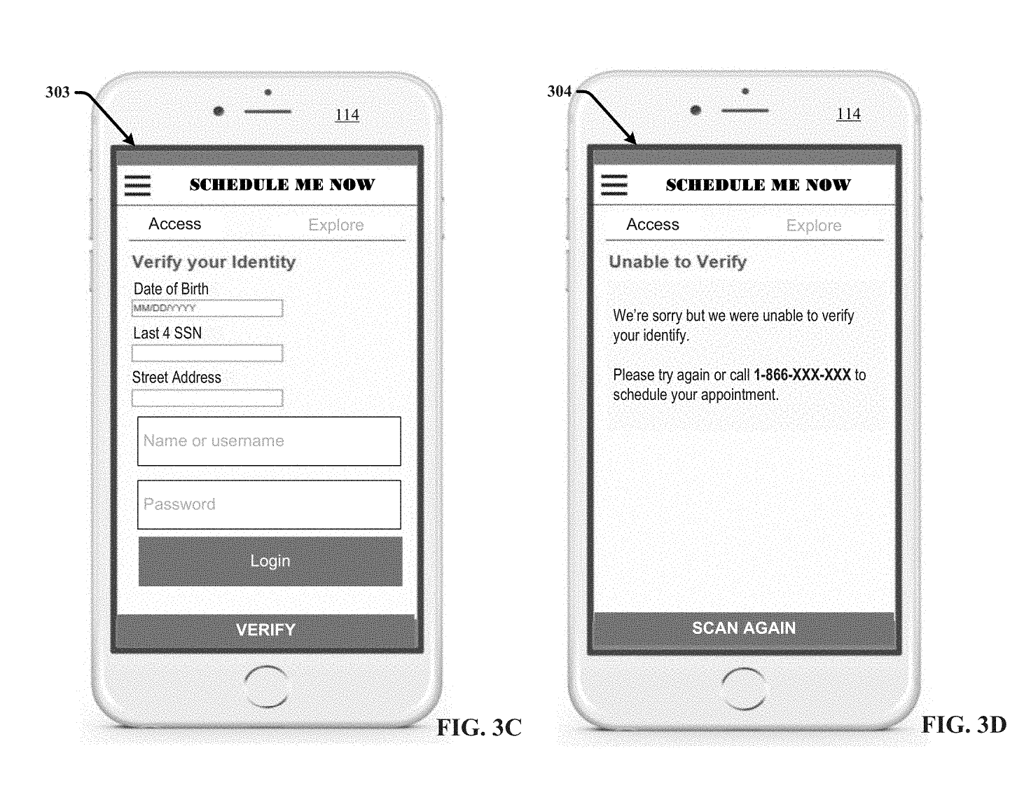

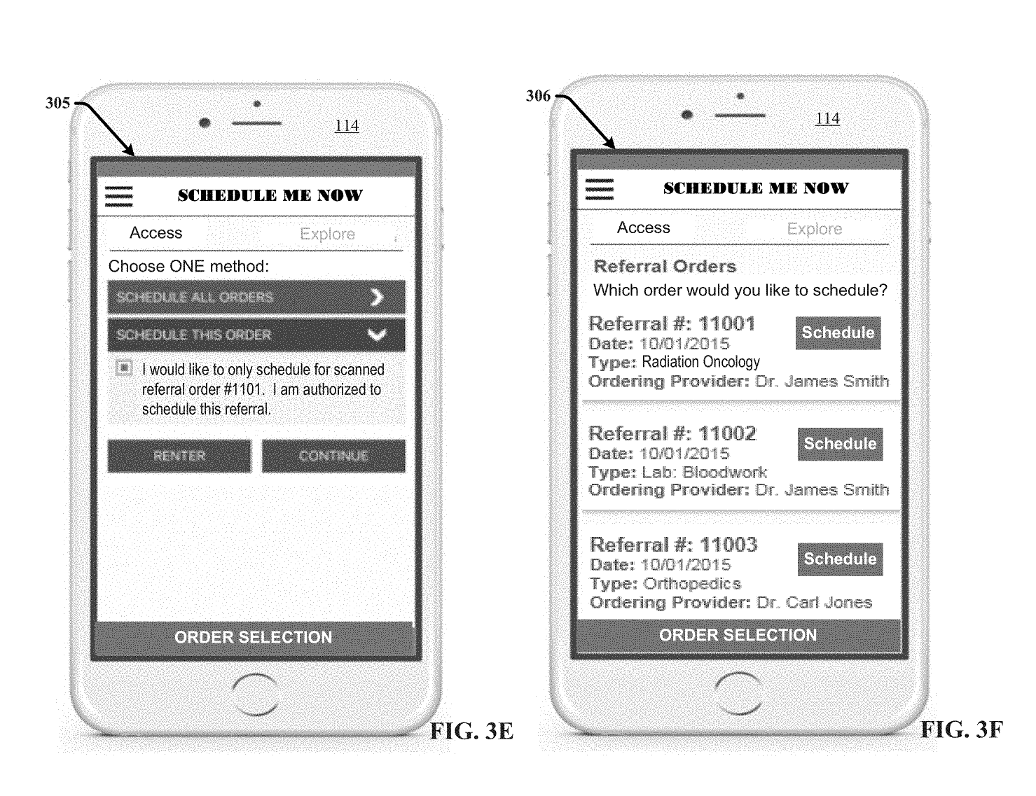

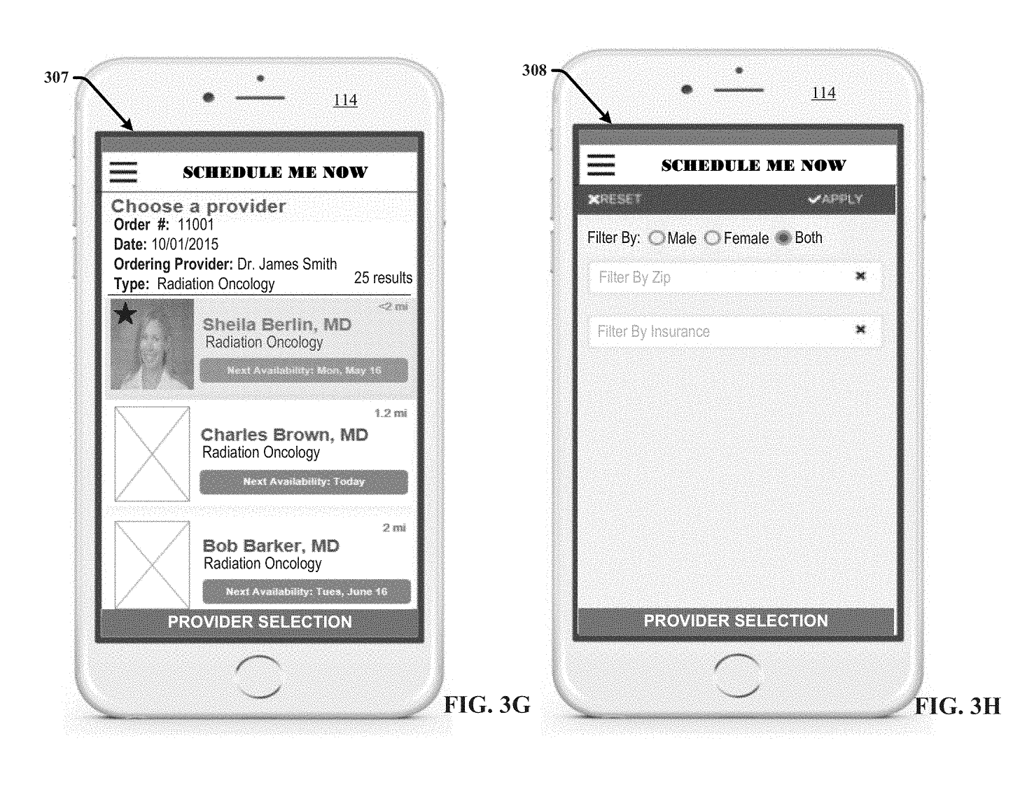

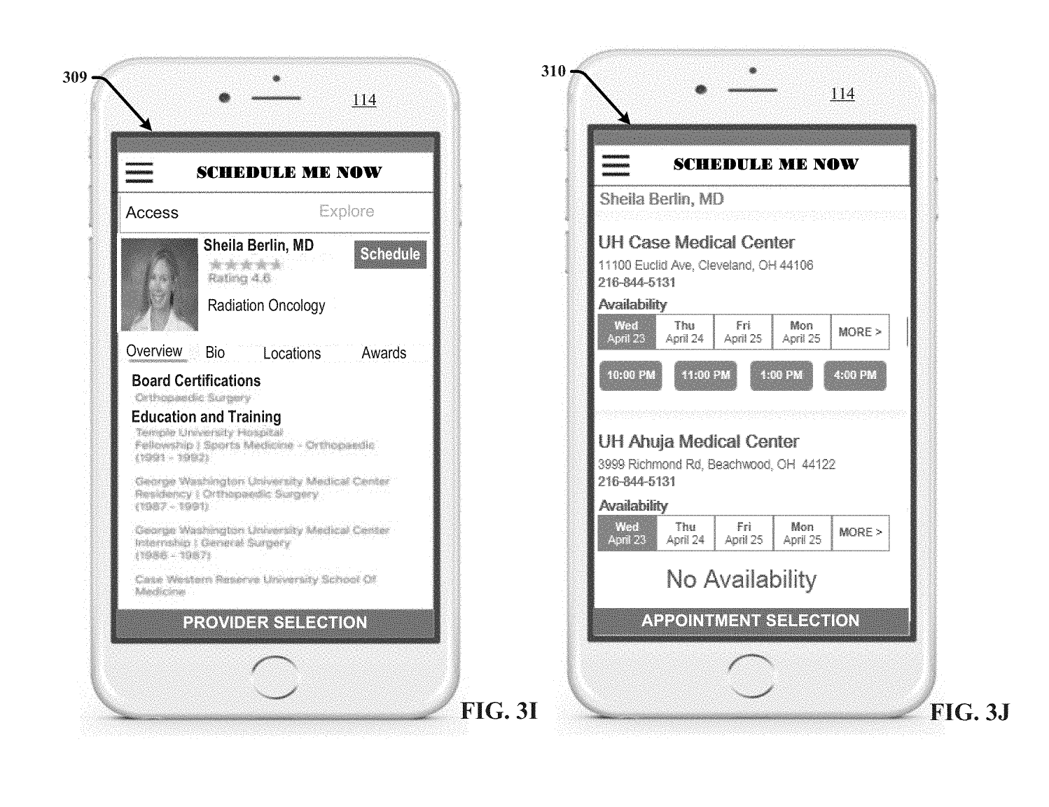

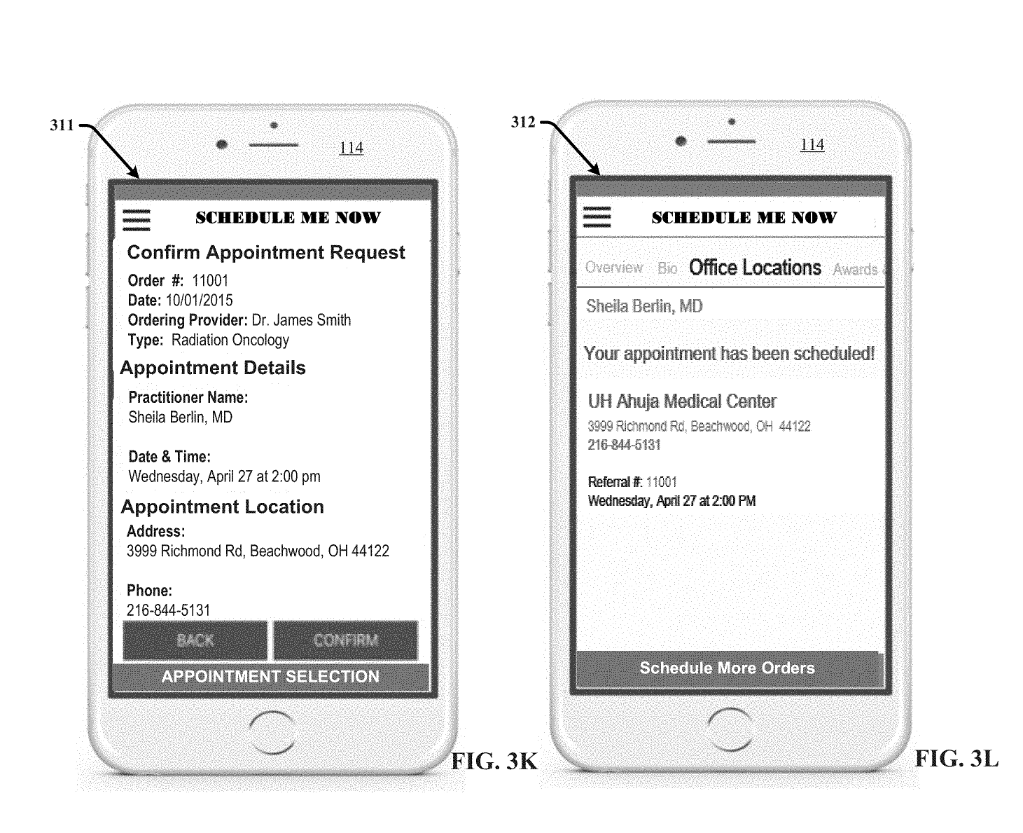

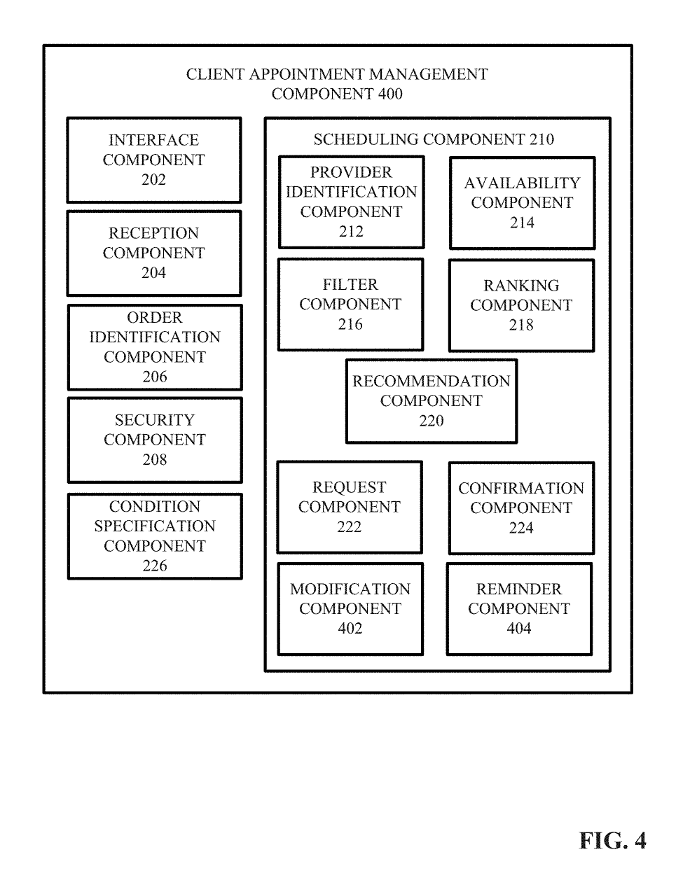

For example, FIGS. 3A-3L presents various GUIs that can be generated by the interface component 202 that facilitate self-scheduling of specialty medical appointments in accordance with various aspects and embodiments described herein. In the embodiment shown, the GUIs are associated with a client application referred to as “Schedule Me Now,” that provides one or more features of the client appointment management component 116. The “Schedule Me Now” application is provided on and/or accessed via a client device (e.g., client device 114) that is a smartphone or tablet type of device. However it should be appreciated the type of client device can vary. Further, in other embodiments, the features and functionalities of the “Schedule Me Now” application can be embodied in website. In this regard, the client device can be configured to access the website via a network (e.g., the Internet) using a suitable browser. FIGS. 3A-3L are discussed in greater detail infra.

With reference back to FIGS. 1 and 2, the reception component 204 can be configured to receive various user input at the client device (e.g., via input component 118) in association with user interaction with the client appointment management component 116. In some embodiments, in order to initiate scheduling a referral appointment, the reception component 204 can be configured to receive input comprising or indicating a referral order identifier. With these embodiments, the patient's referring provider (e.g., the patient's PCP) can provide the patient with a referral order form including a unique referral order identifier for the referral order. For example, in some implementations, when a referring provider issues a referral order for a patient, the referring provider (or an administrator for the referring provider), can generate the referral order by entering the referral order into an electronic system that associates the referral order with the patient's EMR or aEMR. The process of entry of the referral order into the electronic system can result in generation and assignment of a unique referral order identifier to the referral order. The referral order can also include referral order information that indicates or identifies the type of specialist the patient is recommended to see, a service or procedure recommended for the patient, a reason for the referral, a clinical condition of the patient for which the referral order is based, and the like.

In some implementations, the patient can be provided with a printed version of the referral order (e.g., a referral order form or ticket) including the referral order identifier (e.g., a requisition number or order number) and/or a barcode or the like that corresponds to the referral order identifier. In other implementations, the patient can be provided with an electronic copy of the referral order including the referral order identifier (e.g., sent to the patient via email, text, accessed at an electronic user account for the patient, or the like). According to these embodiments, when the patient desires to schedule the specialist appointment for the referral order, the patient can provide the reception component 204 with the referral order identifier. For example, the patient can manually enter the referral order identifier or scan the referral order identifier if the referral order includes a barcode and the patient's client devices includes barcode scanning capabilities.

In one or more embodiments, based on reception of a referral order identifier, the order identification component 206 can be configured to access the patient's EMR in the EMR data store 112 and retrieve the referral order information associated with the referral order. For example, in some implementations, the order identification component 206 can access the EMR data store directly 112. In other implementations, the order identification component 206 can send a request for the order information including the order identifier to the server appointment management component 104. Based on reception of the request, the server appointment management component 104 can be configured to retrieve the order information in the EMR data store and provide the order information to the order identification component. According to these embodiments, because only the patient or an authorized agent of the patient will have the actual printed referral order form or an electronic version of the referral order form including the referral order identifier, unauthorized users will be unable to gain access to the patient's referral order information and schedule referral appointments for the patient.

In some implementations, a patient can have two or more referral orders issued for the patient and associated with the patient's EMR or aEMR in the EMR data store 112. With these implementations, the order identification component 206 can be configured to pull up all referral orders for the patient based on reception of a single referral order identifier for one of the referral orders for the patient. In this regard, based on reception of a referral order identifier for a patient, the order identification component 206 can be configured to identify the referral order corresponding to the referral order identifier as well as an identifier for the patient to whom the referral order is assigned. The order identification component 206 can further determine if the patient has any additional referral orders issued thereto and if so, retrieve the referral order information for the additional referral orders as well as the referral order information associated with the originally provided referral order identifier.

In some embodiments, the client appointment management component 116 can include security component 208 to provide one or more additional security measures to ensure only the patient or an authorized agent of the patient can receive access to the patient's referral order information and schedule referral order appointments for the patient. With the embodiments, the security component 208 can perform an identity verification and/or authorization procedure prior to provision of the referral order information to a user in association with entry of a referral order identifier. For example, in some implementations, in order for a patient to gain access to the patient's EMR or aEMR information, including referral order information, the security component 208 can require the patient to establish a user account that associates a unique identifier for the patient (e.g., name, username, email, phone number, etc.) with secret verification information for the patient (e.g., password, biometric data, or the like). The patient identifier and the verification information can further be associated with the patient's EMR or aEMR. According to these embodiments, in association with reception of an order identifier, the security component 208 can require the user provide the patient identifier and the verification information. The security component 208 can then determine whether the user providing the information is an authorized user if the patient identifier and the verification information both correctly correspond to a particular patient account associated with an EMR or aEMR for a patient, and further if the order identifier is also associated with a referral order for that particular patient EMR or aEMR. Accordingly, if the security component 208 determines that the user is an authorized user, the security component 208 can allow the order identification component 206 to provide the patient with the referral order corresponding to the entered referral order identifier. Otherwise, the security component 208 can deny the user access to the order information.

In one or more alternative embodiments, rather than requiring a user to provide an order identifier in order to retrieve a referral order, all patient referral order can be linked to the patient's EMR or aEMR which can be accessed by the patient or authorized agent of the patient in association with log-in to a patient account that provides the patient access to the EMR or aEMR information. In this regard, the security component 208 can employ the aforementioned identity verification/authorization procedure (e.g., provision of the correct username/password), or a similar identity verification/authorization procedure prior to allowing the user access to the patient's EMR or aEMR information. Once securely authorized and logged-in to a patient's account, the order identification component 206 can be configured to identify all referral orders issued for the patient in association with a user request to view the patient's referral orders.

Once a patient or authorized agent of the patient has been authorized to access and view referral order information associated with a referral order and schedule the referral appointment for the referral order, the scheduling component 210 can facilitate self-scheduling of the associated referral order based on the referral order information. In the embodiment shown, the scheduling component 210 can include provider identification component 212, availability component 214, filter component 216, ranking component 218, recommendation component 220 request component 222 and confirmation component 224.

In one or more embodiments, the provider identification component 212 can be configured to initially determine provider information identifying one or more providers that are qualified to perform the type of medical appointment recommended by a referral order based on the referral order information associated with the referral order and qualification information for the one or more providers. For example, based on information associated with a referral order identifying or indicating the type or specialist the patient is being referred to, the clinical condition of the patient, the reason for the patient's referral or the like, the provider identification component 212 can be configured to query the provider information 108 provided at the medical appointment management server device 102 to identify and/or retrieve information identifying qualified and suitable service providers. For example, if the order information indicates the patient is being referred to see a radiation oncology specialist, the provider identification component 212 can issue a query for all providers recognized in the provider information 108 as being certified radiation oncology specialists. The provider results can further be presented to the user via a GUI generated by the interface component 202 at the client device. The scheduling component 210 can further be configured to schedule the specialist medical appointment for the patient with the server appointment management component 104 based on reception of user input selecting a provider of the one or more providers included in the provider query results.

The availability component 214 configured to determine availability information regarding available appointment slots at which the one or more providers can perform the type or specialist medical appointment indentified by the referral order information. For example, the availability component 214 can be configured to access the scheduling information 106 to identify respective schedules of the service providers to determine respective appointments they have available and information regarding the available appointments. For instance, the availability component 214 can determine, in chronological order, the dates and times of available appointments for the respective service providers identified by the provider identification. In some implementations in which the service providers practice at two or more locations, the availability component 214 can also determine information regarding respective dates and times of available appointments at each location of practice. The availability information for each provider included in the provider results can further be presented to the user via a GUI generated by the interface component 202 at the client device. In some implementations, the GUI can also allow the user to refine, modify and/or re-order the results based on user selected criteria, such as provider availability (e.g., earliest at the top), provider location relative to user location (e.g., the user's current location or a user specified location), and the like. The scheduling component 210 can further be configured to schedule the specialist medical appointment for the patient with the server appointment management component 104 based on reception of user input selecting a particular appointment slot (e.g., at specific date, time and location) with a particular provider.

In various embodiments, rather than providing a user with information regarding all potential service providers that are qualified and capable of performing the type of specialist appointment indicated by a referral order, the filter component 216 can be configured to select a subset of the potential service providers for presenting to the user based on one or more criterion. In some implementations, the one or more criterion can include criterion regarding but are not limited to: service provider availability, service provider location, a schedule of the patient, accepted insurance coverage, and one or more patient preferences.

For example, in some embodiments, the filter component 216 can be configured to select a subset of the qualified service providers based on availability within a defined time frame (e.g., one week, one month, two months, etc.). According to these embodiments, the filter component 216 can examine the availability information for the respective service providers and select only those service providers showing availability within the defined time frame. In some embodiments, the defined time frame can be a default time frame (e.g., one month). In other embodiments, the filter component 216 can be configured to determine the defined time frame based on the referral order information, the patient's scheduled, and/or special circumstances associated with the patient information included in the EMR data store 112. For example, in some implementations, the default time frames can vary depending on the type of specialist appointment or the type of clinical condition affecting the patient. In this regard, the filter component 216 can be configured to determine the default time frame based the type of specialist appointment ordered and/or the clinical condition of the patient. For example, if the patient has been ordered to see a chiropractor regarding back pain, the default time frame can be longer relative to specialist order to see a radiation oncologist regarding a detected tumor in the patient's lymph nodes. In another implementation, the filter component 216 can be configured to determine the time frame based on information associated with the patient (e.g., included in the EMR data store 112) indentifying special circumstances associated with the patient. For example, the special circumstances can state that the patient has a clinical procedure scheduled for a certain date and must see the specialist before that date. In another example, the special circumstances can indicate a level of clinical severity associated with the patient's condition (or another condition affecting the patient), and thus influence the time frame in which the patient should see the specialist recommended in the referral order.

The filter component 216 can be configured to select a subset of the qualified service providers based on locations of the service providers. For example, in some implementations, a patient can be associated with information in their EHR or profile identifying a home location of the patient (e.g., a home address or primary residence address). In accordance with this embodiment the filter component 216 can be configured to select a subset of the qualified service providers based on their practice locations being within a defined distance relative to the patient's home location. In some embodiments, the filter component 216 can receive information identify a patient's current location (e.g., using a global positioning system (GPS) or the like provided by the client device 114) and determine the subset of the service providers based on relative distances between the current location of the patient and the practice locations of the service providers.

Regarding the patient's schedule, in some implementations, the filter component 216 can be configured to determine information regarding when a patient will be available to attend a specialist appointment and where the most convenient location for the patient would be based on information accessible to the filter component 216 regarding the patient's schedule. For example, the filter component 216 can select a subset of providers that have available appointments within a particular time frame that also fit into the patients schedule and do not conflict with other events and appointments scheduled for the patient. In addition, the filter component 216 can select a subset of providers based on the providers having available appointments at times at which the patient is available, and at or near locations where the patient will be located. For instance, the filter component 216 can determine that the patient has an appointment scheduled with a physical therapy specialist from 2:00-3:00 pm at location XYZ. Accordingly the filter component 216, if the patient has been ordered to see a spine specialists, the filter component 216 can be configured to select spine specialists with available appointment near location XYZ on the same day before 2:00 or after 3:00.

In addition, in some implementations, the filter component 216 can be configured to select a subset of available service provider for presenting to the patient based on an insurance provider and/or coverage plan used by the patient and insurance providers/plans accepted by the respective service providers. For example, in the filter component 216 can be configured to select only a subset of the available service providers that accept the patient's insurances.

The filter component 216 can also be configured to select a subset of the service providers for presenting to a patient based on one or more preferences of the patient regarding characteristics of service providers they prefer (or don't prefer) and/or characteristics of appointments they prefer or don't prefer. For example, in some implementations, the filter component 216 can access profile information for the patient that identifies one or more characteristics of preferred providers, such as but not limited to: demographic characteristics (e.g., age, gender, ethnicity, language, etc.), educational characteristics, characteristics regarding performance measures (e.g., patient provided ratings/reviews, peer provided ratings/reviews, etc.), and/or employment history characteristics (e.g., time employed at a particular healthcare organization, time practicing a particular specialty, and the like). In another example, the patient profile information can provide information identifying one or more characteristics or parameters about appointments the patient prefers or doesn't prefer, such as specific times, dates and locations the patient prefers and/or doesn't prefer.

In some embodiments, in addition to or in the alternative to providing automatic filtering of service providers based on one or more of the various criterion discussed above, the filter component 216 can also provide for filtering based on user input. In this regard, the filter component 216 can allow the user to select one or more filter criterion to filter the provider results presented to the user, including the filter criterion discussed above. For example, in association with receiving a list of recommend service providers, the patient can identify one or more filter criteria regarding characteristics of the service providers and/or availability of the service providers and receive only service providers which meet the user defined criteria. For example, in some implementations, the patient can enter a particular date and time, time period, etc., that they would like to book an appointment, and the filter component 216 provide the patient with a list of service providers having available appointments that meet the criteria.

In addition to filtering, the scheduling component 210 can also include ranking component 218 to provide for ranking service providers identified by the provider identification component 212 and/or selected by the filter component 216. In this regard, the ranking component 218 can apply same or similar criterion as the filter component 216 when ranking the respective service providers. In some embodiments, the ranking component can rank or sort the respective service providers based on a single criterion. For example, the single criterion can include but not limited to: most availability within defined a time frame, soonest availability within a defined time frame, closest location to patient home location or other user selected location, closest location to patient current location, and most available appointments that do not conflict with the patient's schedule. The single criterion can also be selected from one or more characteristics associated with the service providers, including a demographic characteristic, an educational level characteristic, a performance measure characteristic, or an employment history characteristic. In some implementations, the ranking component 218 can be configured to automatically rank or sort the service provider based on a particular criterion. In other implementations, the ranking component 218 can be configured to receive user input selecting the ranking or filtering criterion.

In other embodiments, the ranking component 218 can be configured to rank the service providers identified by the provider identification component 212 and/or selected by the filter component 216 based on several criterion to determine the a ranking order for the respective service providers that reflect evaluation of the service provides based on several criterion. In this regard, the ranking component 218 can be configured to associated or determine a score or rank for each service provider that reflects a degree of suitability of the service provider for the patient based on two or more criterion, including but not limited to the criterion above described criterion regarding service provider availability, location, patient schedule, patient insurance, and patient preferences. The recommendation component 220 can further select a subset of the service providers and/or sort the service providers based on their ranking. According to these embodiments, the particular service providers recommended to a patient and/or their order of recommendation will be tailored to each individual patient evaluated by system 100.

In one or more additional embodiments, in addition to identifying and selecting service providers and particular appointment slots for recommending to a patient based on the referral order information included with the patient's referral order in the patient's EMR (and patient preference information in some implementations), the scheduling component 210 can further employ additional user input regarding one or more specific characteristics of the patient's condition. For example, the additional user input can describe one or more characteristics of the patient's condition that are specific to the patient and not included in the patient's referral order information. Such additional information is referred to herein as condition specification information. The provider identification component 212, the filter component 216, the ranking component 218 and/or the recommendation component 220 can further employ the condition specification information to further tailor provider appointment rankings and recommendations. According to these embodiments, the client appointment management component 116 can include condition specification component 226 to facilitate receiving such additional user input.

In some implementations, in association with scanning of a referral order or otherwise selecting a referral order to schedule a specialty medical appointment for, the condition specification component 226 can present the patient with one or more additional qualifying questions determined based on the patient's diagnosis/condition, prior to determining and providing the patient with a list of recommended providers/appointments. In one embodiment, the condition specification component 226 can generate and present the patient with a prompt including the one or more additional questions about the patient's diagnosis/condition and ask the patient to provide answers to the questions. For example, the with respect to a referral order for an orthopedic physician due to knee pain experienced by the patient, the condition specification component 226 can ask the patient for feedback regarding a specific location of the knee pain (e.g. left/right, above the knee/below the knee), a severity of the knee pain, whether the knee pain is sports related/non-sports related, and the like. The answers to these questions can help the provider identification component 212, the filter component 216, the ranking component 218 and the recommendation component 220 refine the list of physicians/appointments recommended to the patient. For instance, in one example, if the patient reports a severity of pain greater than a threshold amount, the filter component 216 can be configured to select only a subset of service providers having available appointments within the next 24 hours. In another example, if the patient reports the knee pain as being sports related, the ranking component 218 can rank be configured to orthopedic physicians specializing in sports injuries higher than general orthopedic physicians.

The specific questions that are presented to the patient by the condition specification component 226 can be determined by the condition specification component 226 based on various factors, including but not limited to, the patient's condition/diagnosis identified in the referral order, the type of physician to which the patient is referred to (as identified in the referral order), and additional information regarding the patients demographics (e.g., age, gender, height, weight, ethnicity), health history/records and/or preferences. In some embodiments, the additional questions can be selected from a database accessible to the condition specification component 226 (e.g., at the client device 114, the medical appointment management server device 102, or at another network accessible location) comprising a plurality of predefined questions that are respectively associated with one or more diagnosis codes/conditions. With these embodiments, the condition specification component 226 can select and present the patient with the appropriate questions based on the patient's diagnosis/condition as defined in the patient's referral order information. In another example, the predefined questions can respectively be associated with one or more or different factors or factor combinations associated with a condition/diagnosis, a type of physician, a patient demographic characteristic (e.g., age, gender, height, weight, ethnicity), a patient health history/record characteristic, and/or a patient preference. With these embodiments, the condition specification component 226 can evaluate a patient's referral order information in view of any additional included in a profile of the patient or the patient's EMR to facilitate selecting the applicable additional questions. In some implementations, depending on the referral order information and the additional information already associated with a patient's EMR and/or profile, additional questions may not be applicable.

In other embodiments, the condition specification component 226 can be configured to present the patient with an open ended question asking the patient if there are any updates regarding the patient's condition/diagnosis and/or if there is any additional information about the patient's condition/diagnosis that the patient would like to report. For example, the condition specification component 226 can be configured to generate a prompt asking the patient whether they have experienced any physical or mental changes in associated with their condition since their last appointment with the refereeing physician.

In various embodiments, the ranking component 218 and the recommendation component 220 can employ one or more machine learning techniques to facilitate tailoring optimal provider recommendations to patients. In addition, the provider identification component 212 and the filter component 216 can employ various machine learning techniques to facilitate identifying suitable providers and appointments for a patient based on the patient's needs and preferences. Further, in some embodiments, the condition specification component 226 can employ one or more machine learning techniques to determine and generate one or more questions for asking a patient in association with gathering condition speciation information for the patient with respect to a particular referral order to further tailor recommended service providers/appointments for fulfilling the referral order.

Machine learning is a type of artificial intelligence (AI) that provides computers with the ability to learn without being explicitly programmed. In order to provide for or aid in the numerous inferences described herein, the ranking component 218 and the recommendation component 220 can examine the entirety or a subset of the data to which it is granted access and can provide for reasoning about or infer states of the system (e.g., system 100 and the like), environment, etc. from a set of observations as captured via events and/or data. An inference can be employed to identify a specific context or action, or can generate a probability distribution over states, for example. The inference can be probabilistic (e.g., the computation of a probability distribution over states of interest can be based on a consideration of data and events). An inference can also refer to techniques employed for composing higher-level events from a set of events and/or data. Such an inference can result in the construction of new events or actions from a set of observed events and/or stored event data, whether or not the events are correlated in close temporal proximity, and whether the events and data come from one or several event and data sources. Various classification (explicitly and/or implicitly trained) schemes and/or systems (e.g., support vector machines, neural networks, expert systems, Bayesian belief networks, fuzzy logic, data fusion engines, etc.) can be employed in connection with performing automatic and/or inferred action in connection with the claimed subject matter.

A classifier can map an input attribute vector, x=(x1, x2, x4, x4, xn), to a confidence that the input belongs to a class, such as by f(x)=confidence(class). Such classification can employ a probabilistic and/or statistical-based analysis (e.g., factoring into the analysis utilities and costs) to prognose or infer an action that a user desires to be automatically performed. A support vector machine (SVM) is an example of a classifier that can be employed. The SVM operates by finding a hyper-surface in the space of possible inputs, where the hyper-surface attempts to split the triggering criteria from the non-triggering events. Intuitively, this makes the classification correct for testing data that is near, but not identical to training data. Other directed and undirected model classification approaches include, e.g., naïve Bayes, Bayesian networks, decision trees, neural networks, fuzzy logic models, and probabilistic classification models providing different patterns of independence can be employed. Classification as used herein also is inclusive of statistical regression that is utilized to develop models of priority.

Once a patient has selected a particular service provider and appointment slot for scheduling a specialist appointment, the request component 221 can send a request to the server appointment management component 104 to enter the appointment into the master scheduling system and/or the internal scheduling system of the service provider. Note that since this is a multi-user system, it's possible that two or more patient can attempt to book the same appointment slot at the same time. In this scenario, the first patient to actually send a request to book the appointment will be successful, and any subsequent requests for the same slot will be denied, (except in the case where a single appointment slot could be double-booked with multiple appointments). Upon successfully entry, the server appointment management component 104 can return an appointment confirmation message to the confirmation component 224 confirming the appointment has been scheduled. The interface component 202 can further present the user with a confirmation message comprising the details of the confirmed appointment. In some embodiments, the scheduling component 210 can further provide the ability to schedule multiple recurring appointments (e.g. every Wednesday for six weeks).

Some of above described features and functionalities of the interface component 202, the reception component 204, the order identification component 206, the security component 208, and the scheduling component 210 are now described with reference to FIGS. 2 and 3A-3L. Repetitive description of like elements employed in respective embodiments is omitted for sake of brevity.

FIG. 3A presents an initial GUI 301 that can be presented to a user in association with opening of the “Schedule Me Now” application at the client device 114. In the embodiment shown, the initial GUI provides an option to schedule a specialist or request an appointment by phone.

FIG. 3B presents an example GUI 302 that can be generated by the interface component 202 in response to selection of the schedule specialist option from GUI 301. As shown in GUI 302, in order to schedule a specialist, the user can be prompted to submit a referral order number by either scanning a barcode (that corresponds to the order number) printed on a referral order form provided to the patient or manually enter the order number. In response to entry of the referral order number, the referral order number is received by the reception component 204 and the order identification component 206 can be configured to look up the order number in the EMR data store to retrieve the order information associated therewith if the order number is valid and associated with a particular patient.

In one embodiment, based on identification of a referral order associated with the referral order number and a patient in the EMR data store 112, the security component 208 can be configured to perform a security procedure that requests the user to verify the user is the patient (or an authorized agent of the patient) before providing the user access to the patient referral order information. For example, FIG. 3C presents an example GUI 303 that can be generated by the interface component 202 in association with such a security procedure in response to entry of the order information via GUI 302 in accordance with this embodiment. As shown in FIG. 3C, the user is prompted to verify their identify by providing verification/authorization information including the user's date of birth, last four digits of their social security number (SSN), street address, name or username and password. The correct verification/authorization information can be associated with the patient information (e.g., or an account for the patient), in the EMR data store 112 or another suitable patient information database. It should be appreciated that these types of verification/authorization information are merely exemplary and that other forms or verification/authorization information can be employed (e.g., biometric information, a PIN number, etc.). Further, it should be appreciated that one or more types of verification/authorization information can be required. In response to reception of the verification/authorization information via GUI 303, the security component 208 can determine whether the verification/authorization information provided is valid for the patient information/account associated with the referral order number. If valid, the security component 208 can provide the user with the patient's referral order information. However, if invalid, the security component 208 can deny the user access to the patient's order information. For example, FIG. 3D presents an example GUI 304 that can be generated by the interface component 202 in response to a determination that the verification/authorization information entered by the user via GUI 303 is invalid.

In another embodiment, the security verification/authorization procedure described above can be removed and the order identification component 206 can be configured to provide the user with the referral order information in response to entry of a valid referral order number that is tied to a referral order associated with a patient/patient account included in the EMR data store 112.

FIG. 3E presents an example GUI 305 that can be generated by the interface component 202 in response to identification of at least one referral order for a patient corresponding to the order number entered via GUI 302 and/or in response to verification of the user's identify as the patient or an authorized agent of the patient. In some implementations the patient/patient account associated with the entered order number (e.g., entered via GUI 302) can be associated with two or more referral orders in the EMR data store 112. In this scenario, in some embodiments, as shown in GUI 305, the order identification component 206 can provide the user with an option to view and schedule all orders associated with the patient/patient account, or to view and schedule only the referral order for referral number entered via GUI 302.

FIG. 3F presents an example GUI 306 that can be generated by the interface component 202 in response to selection of the option to schedule all orders via GUI 305. For example, GUI 306 presents several referral orders (e.g., referral order #11001, #11002, and #11003) issued for the patient. Via GUI 306, the user can select one of the orders to schedule at a time.

FIG. 3G presents an example GUI 307 that can be generated by the interface component 202 in response to selection of referral order #11001 for scheduling via GUI 306. GUI 307 provides a list of service providers for the patient to choose from in order to schedule specialist appointment with based on the selected referral order. The provider results can include some initial information about each provider, such as a picture identifying the provider and availability information identifying their next available appointments. It should be appreciated that this initial information for each provider can vary and is merely exemplary.

For example, the referral order #11001 comprises a referral issued by ordering provider Dr. James Smith for a radiation oncology specialist. Based on the order information associated with the referral order identifying the type of referral as being a referral for radiation oncology, the provider identification component 212 can be configured to identify service providers that are registered radiation oncologists and/or otherwise determined to be able to accommodate the referral order based on keyword matches between one or more the defined terms in the referral order information and one or more defined terms associated with the respective service providers in the provider information 108. The provider identification component 212 can then provide the user with a list of identified, qualifying service providers. In the embodiment shown, the first three of twenty five identified qualifying service providers are listed. The additional service providers can be viewed in response to scrolling up or the like. In some implementations in which the ordering provider recommends a particular service provider in the referral order information, this service provided can be starred, highlighted or otherwise identified in the provider list results. For example, in the embodiment shown, the physician Sheila Berlin, MD is listed first, highlighted and starred. In an aspect, the physician Sheila Berlin, MD is called out in this manner to indicate that she is the service provider recommended to the patient by the ordering provider, Dr. James Smith. In some implementations, providers can also be tagged with a “Best Match” flag indicating that the provider is among the best matched candidate based on metadata or other machine learning matching algorithms.

In various embodiments, the particular providers and the order in which the referring providers are presented to the user in the referral provider list can be controlled by the filter component 216 and the ranking component 218, respectively. For example, in some implementations, the filter component 216 can be configured to automatically select and provide the user with a subset of qualified providers from a set of qualified providers based on one or more criterion, including but not limited to: availability of the respective providers, locations of the respective providers, a location associated with the patient (e.g., a home location or a current location, Best Match), insurance coverage for the patient, and/or one or more preferences of the patient. Likewise, the ranking component 218 can be configured to automatically sort the providers in an order based on a rank determined for the respective providers based on same or similar criterion.

Further, in some embodiments, the filter component 216 and/or the ranking component 218 can allow the user to provide input to control the filtering and/or sorting of the provider results. In this regard, the filter component 216 and/or the ranking component 218 can respectively filter and sort the service provider results based user selected criterion. For example, FIG. 3H presents an example GUI 308 that provides for applying one or more filter and/or sorting criterion for the service provider results. In the embodiment shown, these filter and/or sorting criterion include service provider gender, service provider zip code, and accepted insurance provider.

FIG. 3I presents an example GUI 309 that can be generated by the interface component 202 in response to selection of one of the service providers included in the service provider results from GUI 307. In the embodiment shown, the service provider Sheila Berlin, MD has been selected. As shown in GUI 309, in some implementations, user can be provided with additional information about a selected service provider that can facilitate the user's determination to schedule an appointment with the service provider or not. For example, the additional information can include but is not limited to: a rating received for the provider (e.g., based on patient review), an overview of the provider's board certifications, education and training, a bio, locations at which the service provider practices, awards received, and the like. If the user would like to schedule an appointment with the selected service provider upon review of the additional information about the service provider, the user can select the schedule icon in the upper right hand corner of the GUI.

FIG. 3J presents an example GUI 310 that can be generated by the interface component 202 in response to selection of the schedule icon in GUI 309. In various embodiments, when scheduling an appointment, the user can be provided with information identifying available appointments slots by date, time and location for the service provider. The user can further select a particular available appointment slot at a desired date, time and location.

FIG. 3K presents an example GUI 311 that can be generated by the interface component 202 in response to selection of specific appointment slot from GUI 310. The GUI 311 provides a summary of the selected appointment for the user to review and confirm prior to submitting a request to book the appointment. For example, before submitting a request to schedule particular appointment, the user can be presented with information identifying the referral order information, the appointment details, the appointment location, and the like. If upon review of the request summary information the user would like to proceed with booking the appointment, the user can select the confirm icon in the lower right hand corner of the GUI 311. In one more embodiments, in response to selection of the confirm icon, the request component 222 can send a request to the server appointment management component 104 to enter the appointment into the master scheduling system and/or the internal scheduling system of the service provider. Upon successfully entry, the server appointment management component 104 can return an appointment confirmation message to the confirmation component 224 confirming the appointment has been scheduled. The interface component 202 can further present the user with a confirmation page comprising the details of the confirmed appointment, as shown in FIG. 3L and GUI 312.

In some embodiments, the interface component 202 can provide additional functionality associated with the confirmation page. For example, in some implementations, the confirmation page include an option to save a copy of the confirmation message to the patient's client device and/or add the appointment to the patient's local calendar program on the patient's client device. In another implementation, the confirmation page can include an option to send a copy of the confirmation page to an email account or another electronic messaging account. For example, the email address can be typed in by the patient, selected from a drop down menu, automatically populated into the email address field, or the like. In some implementations, the email address can be an email address employed by the patient and associated with the patient's user account.

FIG. 4 presents another example client appointment management component 400 that facilitates self-scheduling of medical appointments in accordance with various additional embodiments described herein. Client appointment management component 400 can include same or similar features and functionality as client appointment management component 116 with the addition of modification component 402 and reminder component 404 to the scheduling component 210. As described supra, although depicted at the client appointment management component 400, in some embodiments, one or more components of the client appointment management component 400, including one or more components of the scheduling component 210, can be provided at the server appointment management component 104. Repetitive description of like elements employed in respective embodiments described herein is omitted for sake of brevity.

In some implementations, after a patient (or authorized agent of the patient) schedules an appointment with a specialty provider, the patient may desire to cancel the appointment or change the appointment. The modification component 402 can be configured to facilitate making such modifications to a scheduled appointment. For example, in various embodiments, the security component 208 can allow a patient or authorized agent of the patient to access the patient's appointment information, including information regarding scheduled appointment as well as referral order that have yet to be scheduled, based on provision of the correct verification/authorization information and/or successful log-in to a user account associated with the patient. Once logged-in and/or authorized, the patient can selected a scheduled appointment and choose to cancel the appointment or choose modify the appointment by scheduling it at a different time, date and/or location with the same service provider or by scheduling the appointment with a different service provider. In accordance with these embodiments, the request component 222 can generate and send requests to the server appointment management component 104 regarding request to cancel an appointment or modify the appointment and the server appointment management component 104 can update the master scheduling system and scheduling information 106 accordingly.

In accordance with appointment modifications and cancellations, in some embodiments, the availability component 214 can be configured to monitor changes to the scheduling information 108 to identify new appointment openings with the service providers. The availability component 214 can further notify relevant patients regarding the openings. In particular, in some embodiments, a patient can provide input that can be associated with a user account for the patient or otherwise associated with the patients EMR regarding one or more preferred service providers for a particular specialty referral order. The patient can further elect to receive notification regarding new appointment openings with the preferred service provider in association with satisfying the referral order. For example, in one implementation, the patient can schedule a specialty appointment with the patient's second choice provider because their first choice or preferred provider doesn't have any available appointments that fit the patient's schedule within a defined time frame (e.g., their preferred provider has no available appointment this month, for the next two months, etc.). With this implementation, in association with scheduling the appointment with their second choice provider, the patient can provide input identifying their first choice or preferred provider and request to receive updates regarding newly available appointment with their first choice provider. Based on the request, the availability component 214 can further be configured to monitor the scheduling information for newly available appointment with the patient's first choice provider up until their scheduled appointment time arrives. If an appointment with the patient's first choice provider opens up prior to the patient's scheduled appointment, the availability component 214 can be configured to notify the patient regarding the opening. For example, the availability component 214 can provide the patient with a notification via the “Schedule Me Now” application, send an electronic notification or text message, or the like.

In another implementation, if one or more of the patient's preferred providers do not have any available appointments that suit the patient's schedule when the patient is initially attempting to book a referral appointment, the patient can choose to wait to book the appointment and elect to receive notifications regarding any newly available appointments with the one or more identified preferred service providers that for that specialty referral. Still in yet another implementation, the patient can book an appointment with a preferred service provider and also elect to receive notification regarding any earlier appointment openings with that same service provider. In some embodiments, in association with electing to receive notifications regarding new openings with a particular service provider, the patient can further elect an automatic scheduling functionality that directs the scheduling component 210 to automatically book the newly opened appointment with the service provider on behalf of the patient, and then notify the patient regarding the booking thereafter.

The scheduling component 210 can also include a reminder component 404 to provide a patient with reminders associated with scheduled appointments and unscheduled appointments. For example, the reminder component 404 can be configured to generate and provide a patient with an electronic notification, text message, email, or the like, reminding the patient regarding an upcoming appointment at one or more defined points in time prior to the scheduled appointment time (e.g., twenty four hours before, one hour before, etc.). In some implementations, the reminder component 404 can determine based on a current context of the patient, whether the patient is likely to be late to the appointment or miss the appointment entirely (e.g., based on a current location of the patient relative to the appointment location, a current point in time and the scheduled time of the appointment). The reminder component 404 can further send more urgent reminder to the patient based on this context to urge the patient to get to the appointment or notify the service provider regarding their tardiness or inability to make the appointment.

The reminder component 404 can also be configured to provide a patient with reminders regarding unscheduled specialty appointments (e.g., as email, text, push notifications, etc). For example, the reminder component 404 can be configured to monitor the patient EMR or aEMR and the scheduling information 108 to identify referral orders for a patient that have not been scheduled with appointments. In other implementations, the reminder component 404 can be configured to provide patients with notifications regarding new specialty referrals issued for a patient and further monitor if and when the referral orders are scheduled. The reminder component 404 can further send the patient one or more notifications reminding the patient to schedule the specialty referral appointment (e.g., until the appointment is schedule or after passage of a defined period of time). For example, when a specialty referral order is initially generated for a patient and associated with the patients EMR, the reminder component 404 can be configured to generate and send the patient with a notification informing the patient that the specialty referral order has been created and instructing the patient to proceed to schedule the specialty referral appointment. Over time, if the patient does not schedule the specialty referral appointment, the reminder component 404 can be configured to send one or more follow up reminder notifications reminder the patient to schedule the specialty referral appointment. For example, the reminder component 404 can be configured to send a follow up notification at one or more defined points in time following the initial notification if the patient does not schedule the specialty referral appointment (e.g., 24 hours following the initial notification, one week following the initial notification, one month following the initial notification, etc.).

In some implementations, the reminder component 404 can include referral order information in the reminder notifications (including the follow up reminder notifications) sent to a patient that reminds the patient to schedule a referral appointment. For example, the referral order information can include a copy of the referral order, a copy of the referral order number, a copy of the barcode corresponding to the referral order information, and the like. Further in some embodiments, the reminder notification can include a direct link or hyperlink to the referral order information associated with the referral order for which the reminder was sent. In this regard, selection of the link or hyperlink can result in automatically opening the client appointment management application provided by the client appointment management component 116 and accessing the referral order information associated with the link, removing the need for the patient to scan or type in the referral order number. For example, with reference to FIGS. 3C and 3E, in some implementations, selection of the referral order link included in a reminder notification can result in provision of GUI 303 or 305 to the patient at the patient's client device.

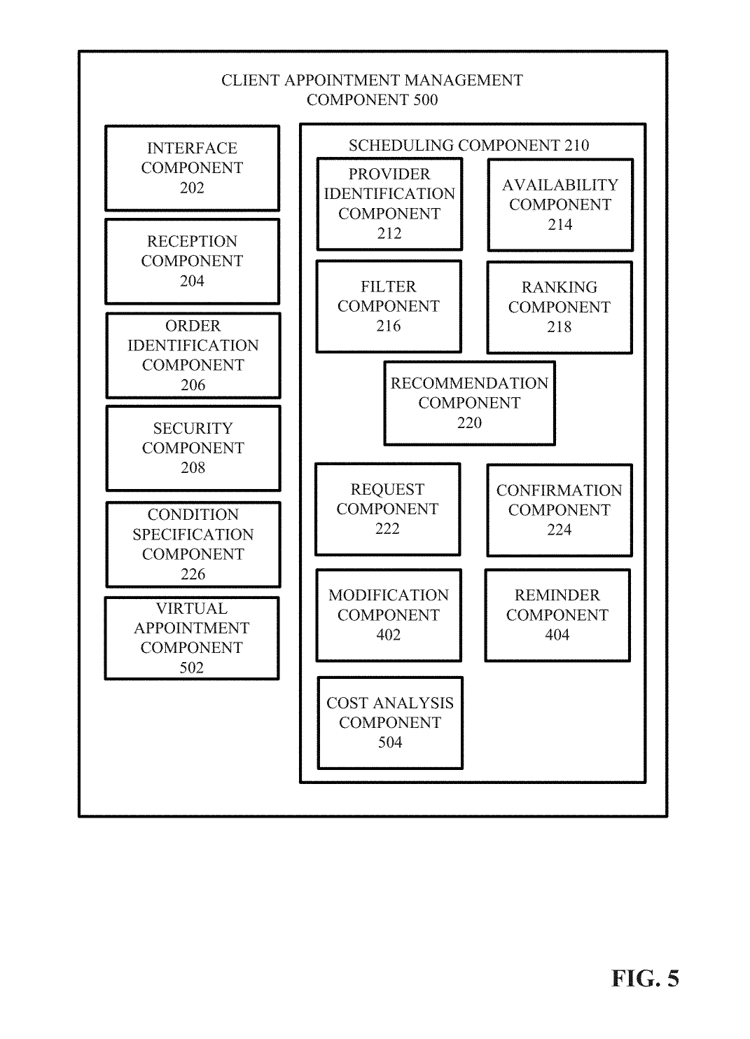

FIG. 5 presents another example client appointment management component 500 that facilitates self-scheduling of medical appointments in accordance with various additional embodiments described herein. Client appointment management component 500 can include same or similar features and functionality as client appointment management component 400 with the addition of virtual appointment component 502 and cost analysis component 504. Repetitive description of like elements employed in respective embodiments described herein is omitted for sake of brevity.

In one or more embodiments, the virtual appointment component 502 can facilitate performing virtual appointments with one or more service providers when appropriate. For example, some types of specialty referral appointments can be performed virtually. Further, some specialty providers may offer virtual appointments at certain times and contexts. According to these embodiments, the provider identification component 212 also identify providers that offer virtual appointments that are suitable for a particular patient based on the referral order information for that patient. Likewise, the filter component 216, the ranking component 218 and the recommendation component 220 can evaluate the virtual appointments in view of one or more criterion (e.g., availability, user schedule, user preferences, user insurance, etc.) to facilitate selecting, ranking and/or recommending the a virtual appointment with a particular provider to a patient.

The cost analysis component 504 can be configured to determine respective costs required for payment by a patient associated with different appointments with different service providers that are capable of satisfying a referral order issued for a patient. In this regard, in association with identifying and/or selecting providers for a patient that can satisfy a referral order, the cost analysis component 504 can determine costs associated with each appointment that the patient would be responsible for based on their insurance plan information and pricing information associated with the different service provider for performance of the specialty appointment recommended by the referral order. The filter component 216, the ranking component 218 and the recommendation component 220 can further employ the cost information when selecting, ranking and/or recommending particular providers and/or appointments for the patient. For example, in some implementations, the filter component 216 can be configured to select only service providers associated with costs less than a threshold amount. In other implementations, the ranking component 218 can rank or sort service provider results based on costs to the patient. Still in other embodiments, the recommendation component can factor in costs to the patient along with other criterion (e.g., availability, location, patient schedule, patient preferences, patient needs, etc.) when determine a recommended subset of providers and/or appointments for recommending to the patient.

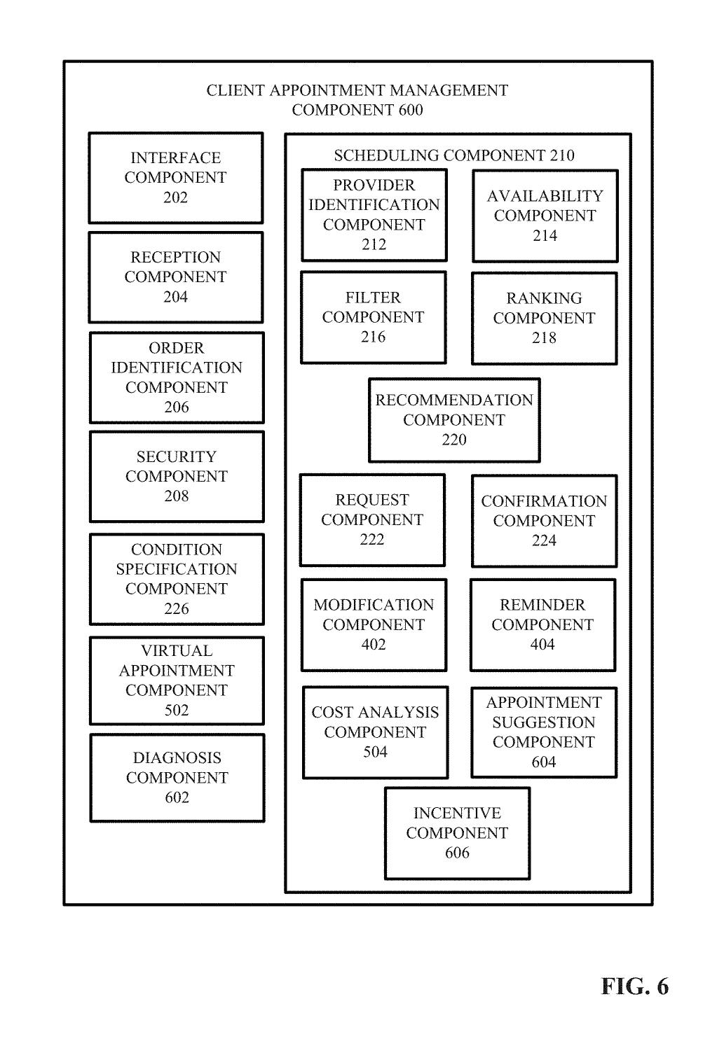

FIG. 6 presents another example client appointment management component 600 that facilitates self-scheduling of medical appointments in accordance with various additional embodiments described herein. Client appointment management component 600 can include same or similar features and functionality as client appointment management component 500 with the addition of diagnosis component 602, appointment suggestion component 604 and incentive component 606. Repetitive description of like elements employed in respective embodiments described herein is omitted for sake of brevity.

In various embodiments described above, the scheduling component 210 can be configured to facilitate scheduling a medical appointment for a patient based on a scheduled referral order generated for the patient by a referring physician. However, in some additional embodiments, the scheduling component 210 can suggest medical appointments for scheduling by a patient based on analysis of medical records of the patient. In this regard, the diagnosis component 602 can be configured to regularly evaluate EMR, aEMR and/or EHR information for the patient to identify and diagnose a condition of the patient for which the patient has not yet seen a specialty physician. In some embodiments, the diagnosis component 602 can employ one or more machine learning techniques to facilitate automatically diagnosing possible patient conditions based various types of information received for a patient and associated with the patient's EMR, aEMR, EHR and the like. The appointment suggestion component 604 can further generate and provide the patient with a notification suggesting and/or instructing the patient to schedule an appointment with a suitable service provider based on the identified condition.

For example, the diagnosis component 602 can determine, based on laboratory reports received for a patient, imaging studies received for a patient, a known condition of the patient, symptoms/side effects reported by a patient, etc., (any of which can be associated in with EMR, aEMR, and/or EHR information for the patient), that a patient likely has a diabetic condition that has not been treated by a specialist. Based on this determination, the appointment suggestion component 604 can be configured to generate and send a notification to the patient recommending the patient schedule an appointment with an endocrinologist due to a possible detected diabetes condition of the patient. The provider identification component 212, the filter component 216, the ranking component 218 and the recommendation component 220 can further facilitate identifying and recommending the best available endocrinologist for the patient's needs. In this case, the patient could be directed to the Schedule Me Now system to book the appointment, even though they do not have a referral order in the EMR system.

In one or more embodiments, the incentive component 606 can be configured to facilitate determining and providing patients with incentives to perform various actions regarding self-scheduling of specialty referral appointments. For example, the incentives can include monetary incentives (e.g., discounts on appointment costs), as well as other suitable incentives (e.g., reduced wait times, additional services, complimentary service upgrades, expedited follow-up appointments, early notifications regarding provider availability, priority scheduling, and the like).

In some embodiments, the incentive component 606 can be configured to provide patients with one or more incentives for booking a specialty referral appointment within a defined window of time after reception of the referral order. For example, the incentive component 606 can provide a patient with an incentive for booking within one hour, one day, one week etc., of a specialty referral appointment after receiving the referral order from their PCP. In some implementations, the incentive component 606 can provide a descending grade of incentives at different points in time (e.g., book within one hour and receive a highest grade incentive, book within a day receive a second grade incentive, book within a week and receive a third grade incentive, etc.). In another embodiment, the incentive component 606 can be configured to provide patients with an incentive for booking a referral order appointment prior to leaving the referring provider's office. In this regard, the incentive component 606 can determine if the time and location of the patient at the time of booking the referral order appointment indicates the patient has not left the referring provider's office to determine whether the incentive applies. In another implementation, the incentive component 606 can determine if the patient books the referral appointment before leaving the referring provider's office based on the device employed by the patient to book the referral appointment (e.g., a tablet computer or kiosk provided at the referring provider's office).

In some embodiments, the incentive component 606 can provide patients with incentives for booking referral appointments with certain service providers, certain appointment times, and/or at certain locations. In particular, some service providers, locations and appointment slots may be more popular than others due to various reasons. For example, a less experienced service provider or newly hired service provider with few reviews may be less popular than other more seasoned service providers. In another example, a particular location may relatively far from most patients or a particular time slot (e.g., late evening) may be undesirable to most patients. In these scenarios, a patient may be deterred from booking the referral appointment altogether because there aren't any upcoming appointment openings with desirable service providers and/or desirable time slots/location. Thus the incentive component 606 can be configured to provide incentives to patients to book with the less popular service providers and appointment times/locations.

Still in other embodiments, the incentive component 606 can be configured to offer patient's with scheduled appointments incentives to trade their appointments in for an alternative appointment with the same provider at a different time/location or with a different provider altogether, based on reception of a request from another patient to take the existing appointment. For example, in some scenarios, a patient with a critical condition may need to book an appointment with a particular service provider and/or within short time frame yet there may be no available appointments. In this situation, the patient and/or the incentive component 606 can issue a request that can be sent to one or more less critical patients with existing appointments to trade their appointment with the critical patient for a later appointment. The incentive component 606 can further offer the less critical patient an incentive for accepting the trade. In other embodiments, the incentive component 606 can allow any patient (critical or not) to initiate a request to pay premium or provide an incentive another patient for offering the other patient their existing appointment. For example, using the incentive component, a patient can issue a notification to other patients with desirable appointment slots with a particular provider, offering an incentive to relinquish their appointment to the patient. If a receiving patient receiving the notification would like to take the incentive and relinquish their appointment, the receiving patient can respond to the notification accordingly and offer up their appointment. The requesting patient can then choose whether to accept the offered appointment slot.

In some implementations, the incentive component 606 can only authorize patients to relinquish their appointment if they book or agree to book another referral appointment to replace the relinquished appointment. In some embodiments, the incentive component 606 can also police patients that employ the subject incentive program to book appointment merely with the intention of receiving incentives and not to ever attend the booked appointments. In this regard, if a particular patient frequently books and relinquishes appointments but does not re-book and attend the re-booked appointments, the patient can be banned from participating in the incentive program or otherwise penalized.

Although various embodiments of system 100 are directed to facilitating scheduling specialty referral appointments that have been issued for a patient, various features and functionalities of system 100 can be extended to scheduling non-specialty referral appointments. In this regard, one or more features and functionalities of system 100 can be employed by a patient (or an authorized agent of the patient) to schedule any type of medical appointment. For example, in some implementations, new patients can employ system 100 to access provider information and their associated scheduling information and to schedule new appointments with PCPs. In association with scheduling new patients, the system can require the patient to provide patient information such as name, address, insurance provider, and the like and/or set up user account/profile with the EMR data store 112 including the patient information.

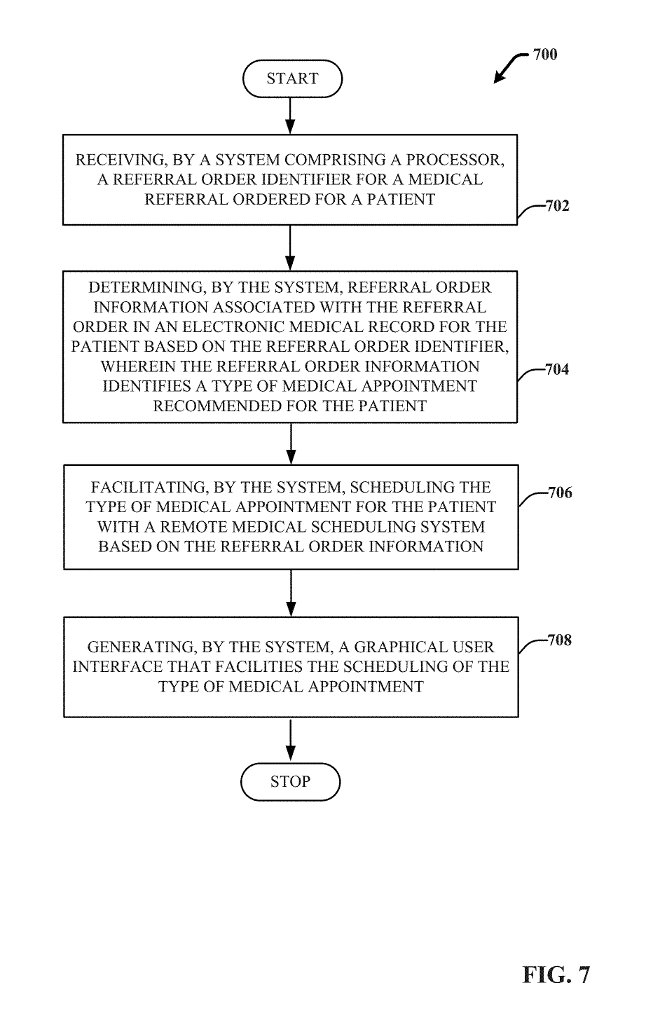

FIGS. 7-9 illustrate various methodologies in accordance with the disclosed subject matter. While, for purposes of simplicity of explanation, the methodologies are shown and described as a series of acts, it is to be understood and appreciated that the disclosed subject matter is not limited by the order of acts, as some acts can occur in different orders and/or concurrently with other acts from that shown and described herein. For example, those skilled in the art will understand and appreciate that a methodology could alternatively be represented as a series of interrelated states or events, such as in a state diagram. Moreover, not all illustrated acts can be required to implement a methodology in accordance with the disclosed subject matter. Additionally, it should be further appreciated that the methodologies disclosed hereinafter and throughout this specification are capable of being stored on an article of manufacture to facilitate transporting and transferring such methodologies to computers.

FIG. 7 provides a high level flow diagram of an example computer-implemented method 700 that facilitates self-scheduling of medical appointments in accordance with various aspects and embodiments described herein. Repetitive description of like elements employed in respective embodiments is omitted for sake of brevity.

At 702, a system (e.g., system 100) comprising a processor, receives a referral order identifier for a medical referral ordered for a patient (e.g., via reception component 204). At 704, the system determines referral order information associated with the referral order in an electronic medical record for the patient based on the referral order identifier (e.g., via order identification component 206), wherein the referral order information identifies a type of medical appointment recommended for the patient. At 706, the system facilitates scheduling the type of medical appointment for the patient with a remote medical scheduling system based on the referral order information (e.g., via scheduling component 210), and at 708, the system generates a GUI that facilities the scheduling of the type of medical appointment (e.g., via interface component 202).

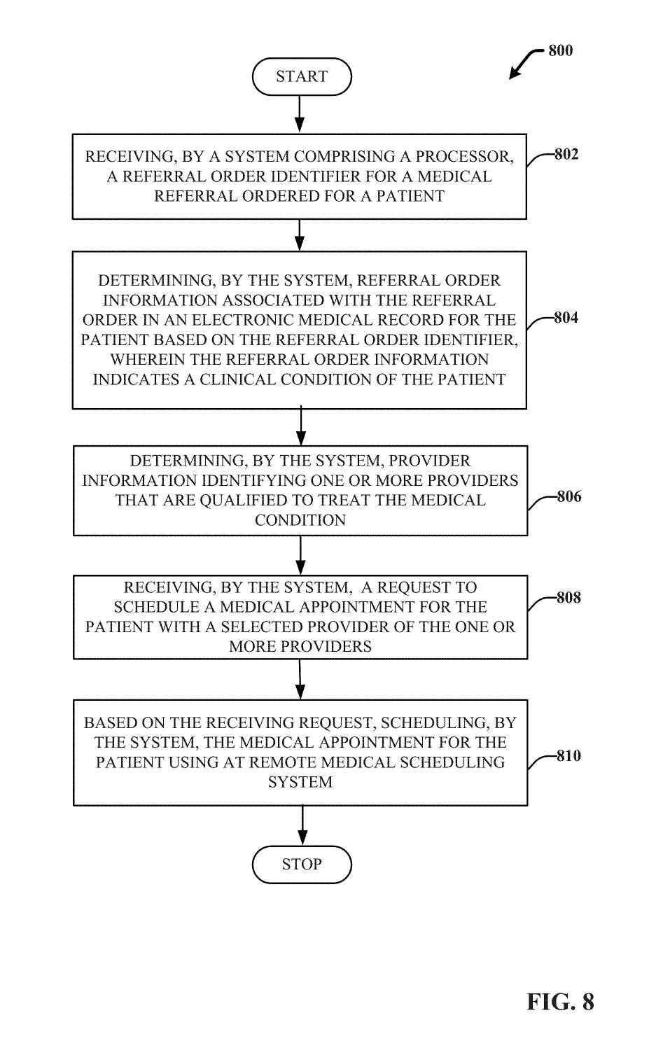

FIG. 8 provides a flow diagram of another example computer-implemented method 800 that facilitates self-scheduling of medical appointments in accordance with various aspects and embodiments described herein. Repetitive description of like elements employed in respective embodiments is omitted for sake of brevity.

At 802, a system (e.g., system 100) comprising a processor, receives a referral order identifier for a medical referral ordered for a patient (e.g., via reception component 204). At 804, the system determines referral order information associated with the referral order in an electronic medical record for the patient based on the referral order identifier (e.g., via order identification component 206), wherein the referral order information indicates a clinical condition of the patient. At 806, the system determines provider information identifying one or more providers that are qualified to treat the medical condition (e.g., via provider identification component 212). At 808, the system receives a request to schedule a medical appointment for the patient with a selected provider of the one or more providers (e.g., via request component 222), and at 810, based on the receiving request, the system schedules the medical appointment for the patient using at remote medical scheduling system (e.g., via scheduling component 210 and server appointment management component 104).

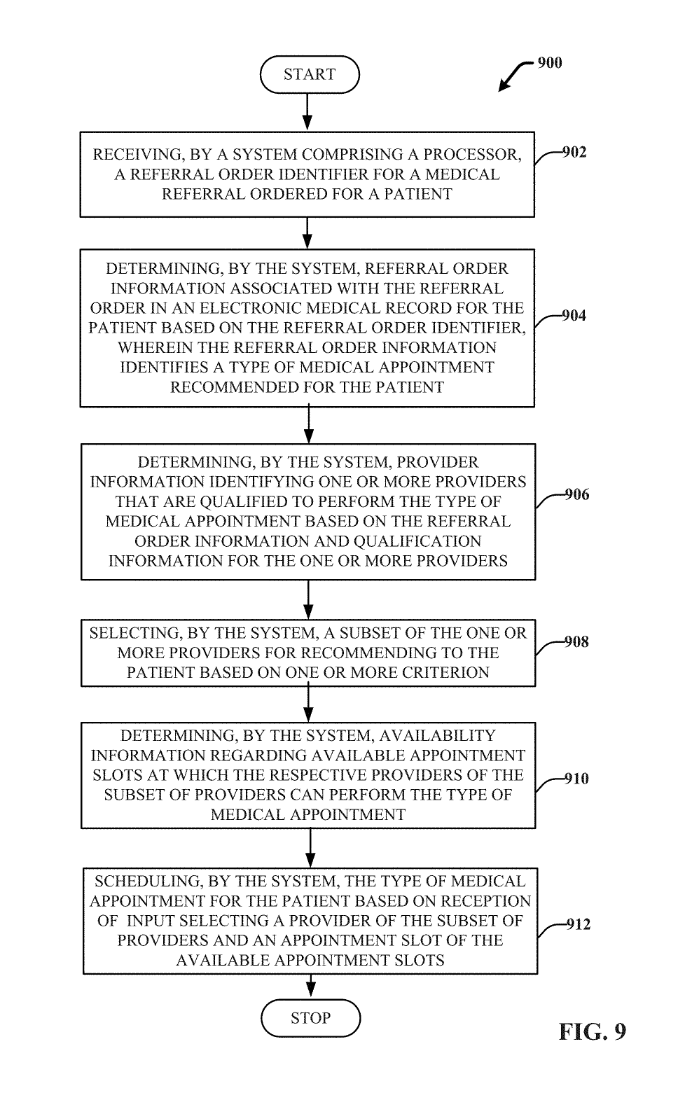

FIG. 9 provides a flow diagram of another example computer-implemented method 900 that facilitates self-scheduling of medical appointments in accordance with various aspects and embodiments described herein. Repetitive description of like elements employed in respective embodiments is omitted for sake of brevity.

At 902, a system (e.g., system 100) comprising a processor, receives a referral order identifier for a medical referral ordered for a patient (e.g., via reception component 204). At 904, the system determines referral order information associated with the referral order in an electronic medical record for the patient based on the referral order identifier (e.g., via order identification component 206), wherein the referral order information identifies a type of medical appointment recommended for the patient. At 906, the system determines provider information identifying one or more providers that are qualified to perform the type of medical appointment based on the referral order information and qualification information for the one or more providers (e.g., via provider identification component 212). At 908, the system selects a subset of the one or more providers for recommending to the patient based on one or more criterion (e.g., via filter component 216 and/or recommendation component 220). At 910, the system, determines availability information regarding available appointment slots at which the respective providers of the subset of providers can perform the type of medical appointment (e.g., via availability component 214). And at 912, the system schedules the type of medical appointment for the patient based on reception of input selecting a provider of the subset of providers and an appointment slot of the available appointment slots (e.g., via scheduling component 210 and server appointment management component 104).

### Example Operating Environments

The systems and processes described below can be embodied within hardware, such as a single integrated circuit (IC) chip, multiple ICs, an application specific integrated circuit (ASIC), or the like. Further, the order in which some or all of the process blocks appear in each process should not be deemed limiting. Rather, it should be understood that some of the process blocks can be executed in a variety of orders, not all of which may be explicitly illustrated in this disclosure.

With reference to FIG. 10, a suitable environment 1000 for implementing various aspects of the claimed subject matter includes a computer 1002. The computer 1002 includes a processing unit 1004, a system memory 1006, a codec 1005, and a system bus 1008. The system bus 1008 couples system components including, but not limited to, the system memory 1006 to the processing unit 1004. The processing unit 1004 can be any of various available suitable processors. Dual microprocessors and other multiprocessor architectures also can be employed as the processing unit 1004.

The system bus 1008 can be any of several types of suitable bus structure(s) including the memory bus or memory controller, a peripheral bus or external bus, and/or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Personal Computer Memory Card International Association bus (PCMCIA), Firewire (IEEE 10104), and Small Computer Systems Interface (SCSI).

The system memory 1006 includes volatile memory 1010 and non-volatile memory 1012. The basic input/output system (BIOS), containing the basic routines to transfer information between elements within the computer 1002, such as during start-up, is stored in non-volatile memory 1012. In addition, according to present innovations, codec 1005 may include at least one of an encoder or decoder, wherein the at least one of an encoder or decoder may consist of hardware, a combination of hardware and software, or software. Although, codec 1005 is depicted as a separate component, codec 1005 may be contained within non-volatile memory 1012. By way of illustration, and not limitation, non-volatile memory 1012 can include read only memory (ROM), programmable ROM (PROM), electrically programmable ROM (EPROM), electrically erasable programmable ROM (EEPROM), or flash memory. Volatile memory 1010 includes random access memory (RAM), which acts as external cache memory. According to present aspects, the volatile memory may store the write operation retry logic (not shown in FIG. 10) and the like. By way of illustration and not limitation, RAM is available in many forms such as static RAM (SRAM), dynamic RAM (DRAM), synchronous DRAM (SDRAM), double data rate SDRAM (DDR SDRAM), and enhanced SDRAM (ESDRAM.

Computer 1002 may also include removable/non-removable, volatile/non-volatile computer storage medium. FIG. 10 illustrates, for example, disk storage 1014. Disk storage 1014 includes, but is not limited to, devices like a magnetic disk drive, solid state disk (SSD) floppy disk drive, tape drive, Jaz drive, Zip drive, LS-70 drive, flash memory card, or memory stick. In addition, disk storage 1014 can include storage medium separately or in combination with other storage medium including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage devices 1014 to the system bus 1008, a removable or non-removable interface is typically used, such as interface 1016.

It is to be appreciated that FIG. 10 describes software that acts as an intermediary between users and the basic computer resources described in the suitable operating environment 1000. Such software includes an operating system 1018. Operating system 1018, which can be stored on disk storage 1014, acts to control and allocate resources of the computer system 1002. Applications 1020 take advantage of the management of resources by operating system 1018 through program modules 1024, and program data 1026, such as the boot/shutdown transaction table and the like, stored either in system memory 1006 or on disk storage 1014. It is to be appreciated that the claimed subject matter can be implemented with various operating systems or combinations of operating systems.

A user enters commands or information into the computer 1002 through input device(s) 1028. Input devices 1028 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 1004 through the system bus 1008 via interface port(s) 1030. Interface port(s) 1030 include, for example, a serial port, a parallel port, a game port, and a universal serial bus (USB). Output device(s) 1036 use some of the same type of ports as input device(s). Thus, for example, a USB port may be used to provide input to computer 1002, and to output information from computer 1002 to an output device 1036. Output adapter 1034 is provided to illustrate that there are some output devices 1036 like monitors, speakers, and printers, among other output devices 1036, which require special adapters. The output adapters 1034 include, by way of illustration and not limitation, video and sound cards that provide a means of connection between the output device 1036 and the system bus 1008. It should be noted that other devices and/or systems of devices provide both input and output capabilities such as remote computer(s) 1038.

Computer 1002 can operate in a networked environment using logical connections to one or more remote computers, such as remote computer(s) 1038. The remote computer(s) 1038 can be a personal computer, a server, a router, a network PC, a workstation, a microprocessor based appliance, a peer device, a smart phone, a tablet, or other network node, and typically includes many of the elements described relative to computer 1002. For purposes of brevity, only a memory storage device 1040 is illustrated with remote computer(s) 1038. Remote computer(s) 1038 is logically connected to computer 1002 through a network interface 1042 and then connected via communication connection(s) 1044. Network interface 1042 encompasses wire and/or wireless communication networks such as local-area networks (LAN) and wide-area networks (WAN) and cellular networks. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL).

Communication connection(s) 1044 refers to the hardware/software employed to connect the network interface 1042 to the bus 1008. While communication connection 1044 is shown for illustrative clarity inside computer 1002, it can also be external to computer 1002. The hardware/software necessary for connection to the network interface 1042 includes, for exemplary purposes only, internal and external technologies such as, modems including regular telephone grade modems, cable modems and DSL modems, ISDN adapters, and wired and wireless Ethernet cards, hubs, and routers.

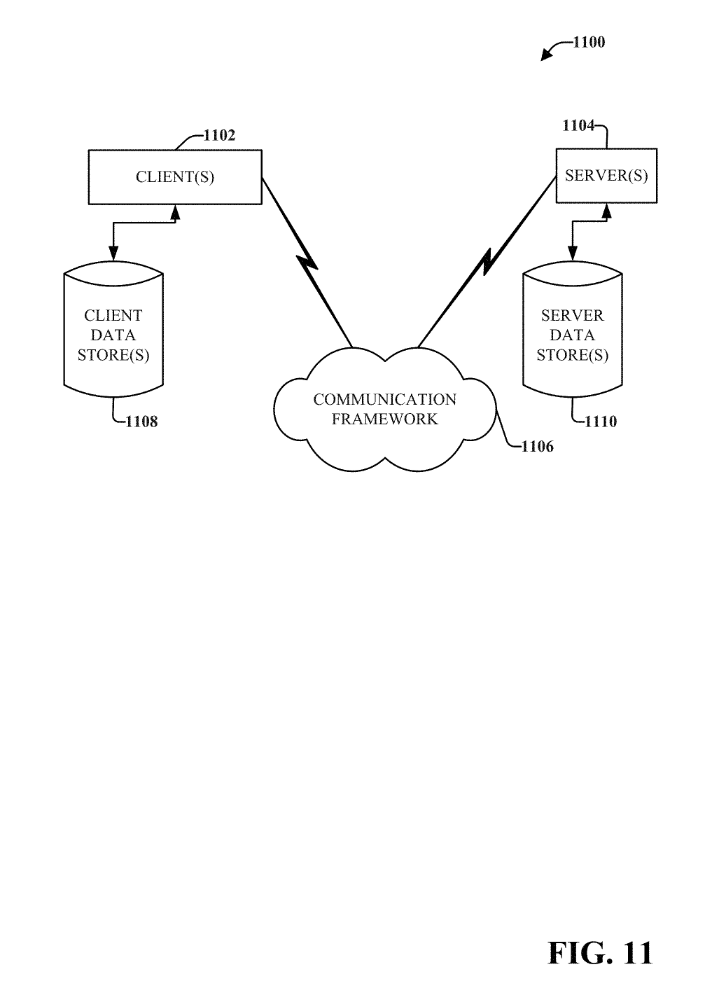

Referring now to FIG. 11, there is illustrated a schematic block diagram of a computing environment 1100 in accordance with this disclosure. The environment 1110 includes one or more client(s) 1102 (e.g. laptops, smart phones, PDAs, media players, computers, portable electronic devices, tablets, and the like). The client(s) 1102 can be hardware and/or software (e.g. threads, processes, computing devices). The system 1100 also includes one or more server(s) 1104. The server(s) 1104 can also be hardware or hardware in combination with software (e.g. threads, processes, computing devices). The servers 1104 can house threads to perform transformations by employing aspects of this disclosure, for example. One possible communication between a client 1102 and a server 1104 can be in the form of a data packet transmitted between two or more computer processes wherein the data packet may include video data. The data packet can include a metadata, e.g. associated contextual information, for example. The system 1100 includes a communication framework 1106 (e.g. a global communication network such as the Internet, or mobile network(s)) that can be employed to facilitate communications between the client(s) 1102 and the server(s) 1104.

Communications can be facilitated via a wired (including optical fiber) and/or wireless technology. The client(s) 1102 include or are operatively connected to one or more client data store(s) 1108 that can be employed to store information local to the client(s) 1102 (e.g. associated contextual information). Similarly, the server(s) 1104 are operatively include or are operatively connected to one or more server data store(s) 1110 that can be employed to store information local to the servers 1104.

In one embodiment, a client 1102 can transfer an encoded file, in accordance with the disclosed subject matter, to server 1104. Server 1104 can store the file, decode the file, or transmit the file to another client 1102. It is to be appreciated, that a client 1102 can also transfer uncompressed file to a server 1104 and server 1104 can compress the file in accordance with the disclosed subject matter. Likewise, server 1104 can encode video information and transmit the information via communication framework 1100 to one or more clients 1102.

The illustrated aspects of the disclosure may also be practiced in distributed computing environments where certain tasks are performed by remote processing devices that are linked through a communications network. In a distributed computing environment, program modules can be located in both local and remote memory storage devices.

Moreover, it is to be appreciated that various components described in this description can include electrical circuit(s) that can include components and circuitry elements of suitable value in order to implement the embodiments of the subject innovation(s). Furthermore, it can be appreciated that many of the various components can be implemented on one or more integrated circuit (IC) chips. For example, in one embodiment, a set of components can be implemented in a single IC chip. In other embodiments, one or more of respective components are fabricated or implemented on separate IC chips.

What has been described above includes examples of the embodiments of the present invention. It is, of course, not possible to describe every conceivable combination of components or methodologies for purposes of describing the claimed subject matter, but it is to be appreciated that many further combinations and permutations of the subject innovation are possible. Accordingly, the claimed subject matter is intended to embrace all such alterations, modifications, and variations that fall within the spirit and scope of the appended claims. Moreover, the above description of illustrated embodiments of the subject disclosure, including what is described in the Abstract, is not intended to be exhaustive or to limit the disclosed embodiments to the precise forms disclosed. While specific embodiments and examples are described in this disclosure for illustrative purposes, various modifications are possible that are considered within the scope of such embodiments and examples, as those skilled in the relevant art can recognize.

In particular and in regard to the various functions performed by the above described components, devices, circuits, systems and the like, the terms used to describe such components are intended to correspond, unless otherwise indicated, to any component which performs the specified function of the described component (e.g. a functional equivalent), even though not structurally equivalent to the disclosed structure, which performs the function in the disclosure illustrated exemplary aspects of the claimed subject matter. In this regard, it will also be recognized that the innovation includes a system as well as a computer-readable storage medium having computer-executable instructions for performing the acts and/or events of the various methods of the claimed subject matter.

The aforementioned systems/circuits/modules have been described with respect to interaction between several components/blocks. It can be appreciated that such systems/circuits and components/blocks can include those components or specified sub-components, some of the specified components or sub-components, and/or additional components, and according to various permutations and combinations of the foregoing. Sub-components can also be implemented as components communicatively coupled to other components rather than included within parent components (hierarchical). Additionally, it should be noted that one or more components may be combined into a single component providing aggregate functionality or divided into several separate sub-components, and any one or more middle layers, such as a management layer, may be provided to communicatively couple to such sub-components in order to provide integrated functionality. Any components described in this disclosure may also interact with one or more other components not specifically described in this disclosure but known by those of skill in the art.

In addition, while a particular feature of the subject innovation may have been disclosed with respect to only one of several implementations, such feature may be combined with one or more other features of the other implementations as may be desired and advantageous for any given or particular application. Furthermore, to the extent that the terms “includes,” “including,” “has,” “contains,” variants thereof, and other similar words are used in either the detailed description or the claims, these terms are intended to be inclusive in a manner similar to the term “comprising” as an open transition word without precluding any additional or other elements.

As used in this application, the terms “component,” “module,” “system,” or the like are generally intended to refer to a computer-related entity, either hardware (e.g. a circuit), a combination of hardware and software, software, or an entity related to an operational machine with one or more specific functionalities. For example, a component may be, but is not limited to being, a process running on a processor (e.g. digital signal processor), a processor, an object, an executable, a thread of execution, a program, and/or a computer. By way of illustration, both an application running on a controller and the controller can be a component. One or more components may reside within a process and/or thread of execution and a component may be localized on one computer and/or distributed between two or more computers. Further, a “device” can come in the form of specially designed hardware; generalized hardware made specialized by the execution of software thereon that enables the hardware to perform specific function; software stored on a computer readable storage medium; software transmitted on a computer readable transmission medium; or a combination thereof.

Moreover, the words “example” or “exemplary” are used in this disclosure to mean serving as an example, instance, or illustration. Any aspect or design described in this disclosure as “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Computing devices typically include a variety of media, which can include computer-readable storage media and/or communications media, in which these two terms are used in this description differently from one another as follows. Computer-readable storage media can be any available storage media that can be accessed by the computer, is typically of a non-transitory nature, and can include both volatile and nonvolatile media, removable and non-removable media. By way of example, and not limitation, computer-readable storage media can be implemented in connection with any method or technology for storage of information such as computer-readable instructions, program modules, structured data, or unstructured data. Computer-readable storage media can include, but are not limited to, RAM, ROM, EEPROM, flash memory or other memory technology, CD-ROM, digital versatile disk (DVD) or other optical disk storage, magnetic cassettes, magnetic tape, magnetic disk storage or other magnetic storage devices, or other tangible and/or non-transitory media which can be used to store desired information. Computer-readable storage media can be accessed by one or more local or remote computing devices, e.g. via access requests, queries or other data retrieval protocols, for a variety of operations with respect to the information stored by the medium.

On the other hand, communications media typically embody computer-readable instructions, data structures, program modules or other structured or unstructured data in a data signal that can be transitory such as a modulated data signal, e.g. a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared and other wireless media.

In view of the exemplary systems described above, methodologies that may be implemented in accordance with the described subject matter will be better appreciated with reference to the flowcharts of the various figures. For simplicity of explanation, the methodologies are depicted and described as a series of acts. However, acts in accordance with this disclosure can occur in various orders and/or concurrently, and with other acts not presented and described in this disclosure. Furthermore, not all illustrated acts may be required to implement the methodologies in accordance with certain aspects of this disclosure. In addition, those skilled in the art will understand and appreciate that the methodologies could alternatively be represented as a series of interrelated states via a state diagram or events. Additionally, it should be appreciated that the methodologies disclosed in this disclosure are capable of being stored on an article of manufacture to facilitate transporting and transferring such methodologies to computing devices. The term article of manufacture, as used in this disclosure, is intended to encompass a computer program accessible from a computer-readable device or storage media.

What is claimed is:

1. A device, comprising:
a memory that stores computer executable components;
a processor that executes computer executable components stored in the memory, wherein the computer executable components comprise:
a reception component configured to receive a referral order identifier for a medical referral ordered for a patient;
a order identification component configured to access an electronic medical record for the patient and retrieve referral order information associated with the referral order based on the referral order identifier, wherein the referral order information identifies a type of medical appointment recommended for the patient;
a scheduling component configured to access a remote medical scheduling system to facilitate scheduling the type of medical appointment for the patient based on the referral order information; and
an interface component configured to generate a graphical user interface that facilities the scheduling of the type of medical appointment.

2. The device of claim 1, wherein the scheduling component comprises a provider identification component configured to determine provider information identifying one or more providers that are qualified to perform the type of medical appointment based on the referral order information and qualification information for the one or more providers.

3. The device of claim 2, wherein the scheduling component is configured to schedule the type of medical appointment for the patient with the remote medical scheduling system based on reception of input selecting a provider of the one or more providers.

4. The device of claim 2, wherein the scheduling component further comprises a filter component configured to select a subset of the one or more providers for recommending to the patient based on one or more criterion.

5. The device of claim 4, wherein the one or more criterion comprise availability of the one or more providers within a defined time frame.

6. The device of claim 4, wherein the one or more criterion comprise respective locations of the one or more providers relative a location associated with the patient.

7. The device of claim 4, wherein the one or more criterion comprise respective types of insurance coverage accepted by the one or more providers relative a type of insurance coverage used by the patient.

8. The device of claim 4, wherein the one or more criterion comprise a preferred characteristic for the subset of medical providers, wherein the preferred characteristic is defined in preference information for the patient.

9. The device of claim 2, wherein the scheduling component further comprises an availability component configured to determine availability information regarding available appointment slots at which the one or more providers can perform the type of medical appointment.

10. The device of claim 9, wherein the scheduling component is configured to schedule the type of medical appointment for the patient with the remote medical scheduling system based on reception of input selecting an appointment slot of the available appointment slots for a selected provider of the one or more providers.

11. The device of claim 1, wherein the reception component is configured to receive the order information in response to scanning, by the device, of a barcode provided on a referral order form.

12. The device of claim 1, wherein the device comprises a mobile phone or a tablet computer and wherein the computer executable components are associated with a mobile application of the device.

13. The device of claim 1, wherein the computer executable components further comprise:
a security component configured to verify an identity of the patient or an authorized agent of the patient prior to authorizing the order identification component to access the electronic medical record for the patient.

14. The device of claim 1, wherein in association with retrieval of the referral order information, the order identification component is further configured to determine whether the patient has received one or more additional referral orders,
wherein based on a determination that the patient has received the one or more additional referral orders, the order identification component is further configured to retrieve additional referral order information associated with the one or more additional referral orders, wherein the scheduling component is further configured to facilitate scheduling one or more additional types of medical appointments for the patient associated with the one or more additional referral orders.

15. The device of claim 1, wherein the computer executable components further comprise:
a reminder component configured generate a notification reminding the patient to schedule the type of medical appointment associated with the referral order based on a determination that the patient has not yet scheduled the type of medical appointment associated with the referral order.

16. A method comprising:
receiving, by a system comprising a processor, a referral order identifier for a medical referral ordered for a patient;
determining, by the system, referral order information associated with the referral order in an electronic medical record for the patient based on the referral order identifier, wherein the referral order information identifies a type of medical appointment recommended for the patient;
facilitating, by the system, scheduling the type of medical appointment for the patient with a remote medical scheduling system based on the referral order information; and
generating, by the system, a graphical user interface that facilities the scheduling of the type of medical appointment.

17. The method of claim 16, wherein the facilitating the scheduling comprises:
determining provider information identifying one or more providers that are qualified to perform the type of medical appointment based on the referral order information and qualification information for the one or more providers; and
scheduling the type of medical appointment for the patient with the remote medical scheduling system based on reception of input selecting a provider of the one or more providers.

18. The method of claim 17, wherein the determining provider information further comprises selecting a subset of the one or more providers for recommending to the patient based on one or more criterion.

19. The method of claim 18, wherein the one or more criterion are based on one or more of: availability of the one or more providers, location of the one or more providers, and insurance accepted by the one or more providers.

20. The method of claim 17, wherein the facilitating further comprises:
determining availability information regarding available appointment slots at which the one or more providers can perform the type of medical appointment, and wherein the scheduling the type of medical appointment further comprises scheduling the type of medical appointment based on reception of additional input selecting an appointment slot of the available appointment slots for a selected provider of the one or more providers.

21. A machine-readable storage medium, comprising executable instructions that, when executed by a processor, facilitate performance of operations, comprising:
receiving a referral order identifier for a medical referral ordered for a patient;
determining referral order information associated with the referral order in an electronic medical record for the patient based on the referral order identifier, wherein the referral order information indicates a clinical condition of the patient;
determining provider information identifying one or more providers that are qualified to treat the clinical condition;
receiving a request to schedule a medical appointment for the patient with a selected provider of the one or more providers; and
based on the receiving request, scheduling the medical appointment for the patient using at remote medical scheduling system.
